SEQUENCE LISTING (24 sequences)

SEQ ID NO: 1 - DNA, 1872 bp, Artificial Sequence
  chemically synthesized
atacgggagccaacaccacgatacccgcttatgaattttaaattaattgtgatcagagcaggtgtgacggatatccgtcacacctgctctgatcacaattaatttaaaattcataagcgggtatcgtggtgttggctcccgtatatacgggagccaacaccaactttcacacatacttgttataccacacgatcttttagagcaggtgtgacggatatccgtcacacctgctctaaaagatcgtgtggtataacaagtatgtgtgaaagttggtgttggctcccgtatatacgggagccaacaccactttgtaactcatttgtagtttgggttgctccccctagagcaggtgtgacggatatccgtcacacctgctctagggggagcaacccaaactacaaatgagttacaaagtggtgttggctcccgtatatacgggagccaacaccatttcccgcttatcttcatccactgcttagcatatgtagagcaggtgtgacggatatccgtcacacctgctctacatatgctaagcagtggatgaagataagcgggaaatggtgttggctcccgtatatacgggagccaacaccaggcactgtatcacaccgtcaagaatgtgatcccctgagagcaggtgtgacggatatccgtcacacctgctctcaggggatcacattcttgacggtgtgatacagtgcctggtgttggctcccgtatatacgggagccaacaccatgtcatttaccttcatcatgacagtgttagtatacgagagcaggtgtgacggatatccgtcacacctgctctaggggatcaaagctatgcgaccatgcgagtggatactggtgttggctcccgtatatacgggagccaacaccagttgccgcctaccttgattattctacattacccgttagagcaggtgtgacggatatccgtcacacctgctctaacgggtaatgtagaataatcaaggtaggcggcaactggtgttggctcccgtatatacgggagccaacaccagtatacatacgaagagttgaaaccaatgcttcgttcagagcaggtgtgacggatatccgtcacacctgctctgaacgaagcattggtttcaactcttcgtatgtatactggtgttggctcccgtatatacgggagccaacaccataccccgaatggctgttttcagtaccaatatgactcagagcaggtgtgacggatatccgtcacacctgctctgagtcatattggtactgaaaacagccattcggggtatggtgttggctcccgtatatacgggagccaacaccactgtcacgatcgtcgttccttttaatccttgtgtctagagcaggtgtgacggatatccgtcacacctgctctagacacaaggattaaaaggaacgacgatcgtgacagtggtgttggctcccgtatatacgggagccaacaccactggacactgaccctcgcactagctttctgacgggtagagcaggtgtgacggatatccgtcacacctgctctacccggccgaagaatagtgctcggtacttagtcgcgtggtgttggctcccgtatatacgggagccaacaccatttggactttaaatagtggactccttctttgtctcgagagcaggtgtgacggatatccgtcacacctgctctcgagacaaagaaggagtccactatttaaagtccaaatggtgttggctcccgtatatacgggagccaacaccatcatttgcaaatatgaattccacttaaagaaattcaagagcaggtgtgacggatatccgtcacacctgctcttgaatttctttaagtggaattcatatttgcaaatgatggtgttggctcccgtat

SEQ ID NO: 2 - DNA, 718 bp, Artificial Sequence
  chemically synthesized
atacgggagccaacaccatcctaactggtctaatttttgctgttaccgatcccgagagcaggtgtgacggatatccgtcactcctgctctcgggatcggtaacagcaaaaattagaccagttaggatggtgttggctcccgtatatacgggagccaacaccagcctgacgaaaaaattttatcactaagtgatacgcaagagcaggtgtgacggatatccgtcacacctgctcttgcgtatcacttagtgataaaattttttcgtcaggctggtgttggctcccgtatatacgggagccaacaccagacctacttcagaaacggaaatgttcttagccgtcagagcaggtgtgacggatatccgtcacacctgctctgacggctaagaacatttccgtttctgaagtaggtctggtgttggctcccgtatatacgggagccaacaccaggccaacgaaactcctactacatataatgcttatgcagagcaggtgtgacggatatccgtcacacctgctctgcataagcattatatgtagtaggagtttcgttggcctggtgttggctcccgtatatacgggagccaacaccatcctaactggtctaatttttgctgttaccgatcccgagagcaggtgtgacggatatccgtcacacctgctctcgggatcggtaacagcaaaaattagaccagttaggatggtgttggctcccgtat

SEQ ID NO: 3 - DNA, 60 bp, Artificial Sequence
  chemically synthesized
catccgtcacacctgctctggccactaacatggggaccaggtggtgttggctcccgtatc

SEQ ID NO: 4 - DNA, 242 bp, Artificial Sequence
  chemically synthesized
catccgtcacacctgctctgctatcacatgcctgctgaagtggtgttggctcccgtatcacatccgtcacacctgctctggggatgtgtggtgttggctcccgtatcaagggcgaattctcatccgtcacacctgctctgatcagggaagacgccaacacgtggtgttggctcccgtatcacatccgtcacacctgctctgggtggtgttggctcccgtatcaagggcgaattctgcagata

SEQ ID NO: 5 - DNA, 372 bp, Artificial Sequence
  chemically synthesized
catccgtcacacctgctctggggagggtggcgcccgtctcggtggtgttggctcccgtatcacatccgtcacacctgctctgggatagggtctcgtgctagatgtggtgttggctcccgtatcacatccgtcacacctgctctggaccggcgcttattcctgcttgtggtgttggctcccgtatcacatccgtcacacctgcyctggagctgatattggatggtccggtggtgttggctcccgtatcacatccgtcacacctgcycygcccagagcaggtgtgacggatgtggtgttggctcccgtatcacatccgtcacacctgcycygccggaccatccaatatcagctgtggtgttggctcccgtatca

SEQ ID NO: 6 - DNA, 864 bp, Artificial Sequence
  chemically synthesized
atacgggagccaacaccattaaatcaattgtgccgtgttggtcttgtctcatcgagagcaggtgtgacggatatccgtcacacctgctctcgatgagacaagaccaacacggcacaattgatttaatggtgttggctcccgtatatacgggagccaacaccatttttattatcggtatgatcctacgagttcctcccaagagcaggtgtgacggatatccgtcacacctgctcttgggaggaactcgtaggatcataccgataataaaaatggtgttggctcccgtatatacgggagccaacaccaccgtatatcttattatgcacagcatcacgaaagtgcagagcaggtgtgacggatatccgtcacacctgctctgcactttcgtgatgctgtgcataataagatatacggtggtgttggctcccgtatatacgggagccaacaccattaacgttaagcggcctcacttcttttaatcctttcagagcaggtgtgacggatatccgtcacacctgctctgaaaggattaaaagaagtgaggccgcttaacgttaatggtgttggctcccgtatatccgtcacacctgctctaatatagaggtattgctcttggacaaggtacagggatggtgttggctcccgtatatacgggagccaacaccatccctgtaccttgtccaagagcaatacctctatattagagcaggtgtgacggatatacgggagccaacaccattaacgttaagcggcctcacttcttttaatcctttcagagcaggtgtgacggatatccgtcacacctgctctgaaaggattaaaagaagtgaggccgcttaacgttaatggtgttggctcccgtat

SEQ ID NO: 7 - DNA, 574 bp, Artificial Sequence
  chemically synthesized
atccgtcacacctgctctaattaggatacggggcaacagaacgagaggggggaatggtgttggctcccgtatatccgtcacacctgctctcggaccaggtcagacaagcacatcggatatccggctggtgttggctcccgtatatccgtcacacctgctctaattaggatacggggcaacagaacgagaggggggaatggtgttggctcccgtatatccgtcacacctgctcttgagtcaaagagtttagggaggagctaacataacagtggtgttggctcccgtatatccgtcacacctgctctaacaacaatgcatcagcgggctgggaacgcatgcggtggtgttggctcccgtatatccgtcacacctgctctgaacaggttataagcaggagtgatagtttcaggatctggtgttggctcccgtatatccgtcacacctgctctcggcggctcgcaaaccgagtggtcagcacccgggttggtgttggctcccgtatatccgtcacacctgctctgcgcaagacgtaatccacaagaccgtgaaaacatagtggtgttggctcccgtat

SEQ ID NO: 8 - DNA, 574 bp, Artificial Sequence
  chemically synthesized
atacgggagccaacaccattcccccctctcgttctgttgccccgtatcctaattagagcaggtgtgacggatatacgggagccaacaccagccggatatccgatgtgcttgtctgacctggtccgagagcaggtgtgacggatatacgggagccaacaccattcccccctctcgttctgttgccccgtatcctaattagagcaggtgtgacggatatacgggagccaacaccactgttatgttagctcctccctaaactctttgactcaagagcaggtgtgacggatatacgggagccaacaccaccgcatgcgttcccagcccgctgatgcattgttgttagagcaggtgtgacggatatacgggagccaacaccagatcctgaaactatcactcctgcttataacctgttcagagcaggtgtgacggatatacgggagccaacaccaacccgggtgctgaccactcggtttgcgagccgccgagagcaggtgtgacggatatacgggagccaacaccactatgttttcacggtcttgtggattacgtcttgcgcagagcaggtgtgacggat

SEQ ID NO: 9 - DNA, 432 bp, Artificial Sequence
  chemically synthesized
atccgtcacacctgctctaggcgtagtgactaagtcgcgcgaaaatcacagcattggtgttggctcccgtatatccgtcacacctgctctcagcggcagctatacagtgagaacggactagtgcgttggtgttggctcccgtatatccgtcacacctgctctggcaaataatactagcgatgatggatctggatagactggtgttggctcccgtatatccgtcacacctgctctgggggtgcgacttagggtaagtgggaaagacgatgctggtgttggctcccgtatatccgtcacacctgctctcaagaggagatgaaccaatcttagtccgacaggcggtggtgttggctcccgtatatccgtcacacctgctctggcccggaattgtcatgacgtcacctacacctcctgtggtgttggctcccgtat

SEQ ID NO: 10 - DNA, 432 bp, Artificial Sequence
  chemically synthesized
atacgggagccaacaccaatgctgtgattttcgcgcgacttagtcactacgcctagagcaggtgtgacggatatacgggagccaacaccaacgcactagtccgttctcactgtatagctgccgctgagagcaggtgtgacggatatacgggagccaacaccagtctatccagatccatcatcgctagtattatttgccagagcaggtgtgacggatatacgggagccaacaccagcatcgtctttcccacttaccctaagtcgcacccccagagcaggtgtgacggatatacgggagccaacaccaccgcctgtcggactaagattggttcatctcctcttgagagcaggtgtgacggatatacgggagccaacaccacaggaggtgtaggtgacgtcatgacaattccgggccagagcaggtgtgacggat

SEQ ID NO: 11 - DNA, 242 bp, Artificial Sequence
  chemically synthesized
gatacgggagccaacaccacccgtatcgttcccaatgcactcagagcaggtgtgacggatgcatccgtcacacctgctctgagtgcattgggaacgatacgggtggtgttggctcccgtatggatacgggagccaacaccacgttcccatacaagttactgacagagcaggtgtgacggatgcatccgtcacacctgctctgtcagtaacttgtatgggaacgtggtgttggctcccgtatc

SEQ ID NO: 12 - DNA, 575 bp, Artificial Sequence
  chemically synthesized
atccgtcacccctgctctcgtcgctatgaagtaacaaagataggagcaatcgggtggtgttggctcccgtatatccgtcacacctgctctaacgaagactgaaaccaaagcagtgacagtgctgaatggtgttggctcccgtatatccgtcacacctgctctcggtgacaatagctcgatcagcccaaagtcgtcagatggtgttggctcccgtatatccgtcacacctgctctaacgaaatagaccacaaatcgatactttatgttattggtgttggctcccgtatatccgtcacacctgctctgtcgaatgctctgcctggaagagttgttagcagggatggtgttggctcccgtatatccgtcacacctgctcttaagccgaggggtaaatctaggacaggggtccatgatggtgttggctcccgtatatccgtcacacctgctctactggccggctcagcatgactaagaaggaagttatgtggtgttggctcccgtatatccgtcacacctgctctggtacgaatcacaggggatgctggaagcttggctcttggtgttggctcccgtat

SEQ ID NO: 13 - DNA, 575 bp, Artificial Sequence
  chemically synthesized
atacgggagccaacaccacccgattgctcctatctttgttacttcatagcgacgagagcaggggtgacggatatacgggagccaacaccattcagcactgtcactgctttggtttcagtcttcgttagagcaggtgtgacggatatacgggagccaacaccatctgacgactttgggctgatcgagctattgtcaccgagagcaggtgtgacggatatacgggagccaacaccaataacataaagtatcgatttgtggtctatttcgttagagcaggtgtgacggatatacgggagccaacaccatccctgctaacaactcttccaggcagagcattcgacagagcaggtgtgacggatatacgggagccaacaccatcatggacccctgtcctagatttacccctcggcttaagagcaggtgtgacggatatacgggagccaacaccacataacttccttcttagtcatgctgagccggccagtagagcaggtgtgacggatatacgggagccaacaccaagagccaagcttccagcatcccctgtgattcgtaccagagcaggtgtgacggat

SEQ ID NO: 14 - DNA, 144 bp, Artificial Sequence
  chemically synthesized
atacgggagccaacaccattaaatcaattgtgccgtgttcctcttgtctcatcgagagcaggttgtacggatatccgtacaacctgctctcgatgagacaagaggaacacggcacaattgatttaatggtgttggctcccgtat

SEQ ID NO: 15 - DNA, 400 bp, Artificial Sequence
  chemically synthesized
atacgggagccaacaccagcagtcaagaagttaagagaaaaacaattgtgtataagagcaggtgtgacggatatccgtcacacctgctcttatacacaattgtttttctcttaacttcttgactgctggtgttggctcccgtatatccgtcacacctgctctgcgccacaagattgcggaaagacacccggggggcttggtgttggctcccgtatatacgggagccaacaccaagccccccgggtgtctttccgcaatcttgtggcgcagagcaggtgtgacggatatccgtcacacctgctctggccttatgtaaagcgttgggtggtgttggctcccgtatatacgggagccaacaccacccaacgctttacataaggccagagcaggtgtgacggat

SEQ ID NO: 16 - DNA, 241 bp, Artificial Sequence
  chemically synthesized
catccgtcacacctgctctggttcgccccggtcaaggagagtggtgttggctcccgtatcgatacgggagccaacaccactctccttgaccggggcgaaccagagcaggtgtgacggatgcatccgtcacacctgctctggataagatcagcaacaagttagtggtgttggctcccgtatcgatacgggagccaacaccactaacttgttgctgatcttatcagagcaggtgtgacggatg

SEQ ID NO: 17 - DNA, 490 bp, Artificial Sequence
  chemically synthesized
atccgtcacacctgctctccgcacgtaggaccactttggtacacgctcccgtagtggtgttggctcccgtatatccgtcacacctgctctacggatgaacgaagattttaaagtcaagctaatgcatggtgttggctcccgtatatccgtcacacctgctctgtagtgaagagtccgcagtccacgctgttcaactcatggtgttggctcccgtatatccgtcacacctgctctaccggctggcacggttatgtgtgacgggcgaagatatggtgttggctcccgtatatccgtcacacctgctctaccggctggcacggttatgtgtgacgggcgaagatatggtgttggctcccgtatatccgtcacacctgctctgcgtgtggagcgcctaggtgagtggtgttggctcccgtatatccgtcacacctgctctgatgtccctttgaagagttccatgacgctggctccttggtgttggctcccgtat

SEQ ID NO: 18 - DNA, 490 bp, Artificial Sequence
  chemically synthesized
atacgggagccaacaccactacgggagcgtgtaccaaagtggtcctacgtgcggagagcaggtgtgacggatatacgggagccaacaccatgcattagcttgactttaaaatcttcgttcatccgtagagcaggtgtgacggatatacgggagccaacaccatgagttgaacagcgtggactgcggactcttcactacagagcaggtgtgacggatatacgggagccaacaccatatcttcgcccgtcacacataaccgtgccagccggtagagcaggtgtgacggatatacgggagccaacaccatatcttcgcccgtcacacataaccgtgccagccggtagagcaggtgtgacggatatacgggagccaacaccactcacctaggcgctccacacgcagagcaggtgtgacggatatacgggagccaacaccaaggagccagcgtcatggaactcttcaaagggacatcagagcaggtgtgacggat

SEQ ID NO: 19 - DNA, 1567 bp, Artificial Sequence
  chemically synthesized
atacgggagccaacaccatagtgttgggccaatacggtaacgtgtccttggagagcaggtgtgacggatatccgtcacacctgctctccaaggacacgttaccgacgaattggcccaacactatggtgttggctcccgtatatacgggagccaacaccacacatacgagttatctcgagtagagcatgttttgccagagcaggtgtgacggatatccgtcacacctgctctggcaaaacatgctctactcgagataactcgtatgtgtggtgttggctcccgtatatacgggagccaacaccaggccatctattgttcgtttttctatttatctcacccagagcaggtgtgacggatatccgtcacacctgctctgggtgagataaatagaaaaacgaacaatagatggcctggtgttggctcccgtatatacgggagccaacaccacacatacgagttatctcgagtagagcatgttttgccagagcaggtgtgacggatatccgtcacacctgctctggcaaaacatgctctactcgagataactcgtatgtgtggtgttggctcccgtatatacgggagccaacaccatccatagctcatctataccctcttccgagtcccaccagagcaggtgtgacggatatccgtcacacctgctctggtgggactcggaagagggtatagatgagctatggatggtgttggctcccgtatatacgggagccaacaccagagcaggtgtgacggatagtgacggatgcagagcaggtgtgacggatatccgtcacacctgctctgcatccgtcactatccgtcacacctgctctggtgttggctcccgtatatacgggagccaacaccaccttatgacgcctcagtaccacatcgtttagtctgtagagcaggtgtgacggatatccgtcacacctgctctacagactaaacgatgtggtactgaggcgtcataaggtggtgttggctcccgtatatacgggagccaacaccacccgtttttgatctaatgaggatacaatattcgtctagagcaggtgtgacggatatccgtcacacctgctctagacgaatattgtatcctcattagatcaaaaacgggtggtgttggctcccgtatatacgggagccaacaccatcgagctccttggccccgttaggattaacgtgatgtagagcaggtgtgacggatatccgtcacacctgctctacatcacgttaatcctaacggggccaaggagctcgatggtgttggctcccgtatatacgggagccaacaccatcagaaccaaatatacatcttcctatgatatggtggagagcaggtgtgacggatatccgtcacacctgctctccaccatatcataggaagatgtatatttggttctgatggtgttggctcccgtatatacgggagccaacaccacacgattgctcctctcattgttacttcatagcgacgagagcaggtgtgacggatatccgtcacacctgctctcgtcgctatgaagtaacaatgagaggagcaatcgtgtggtgttggctcccgtat

SEQ ID NO: 20 - DNA, 576 bp, Artificial Sequence
  chemically synthesized
gatacgggacgacaccacactatgggtcgtttagcatcaaggctagccaagccagcagaggtgtggtgaatgcattcaccacacctctgctggcttggctagccttgatgctaaacgacccatagtgtggtgtcgtcccgtatccattcaccacacctctgctggaggaggaagtggtctggagttacttgacatagtgtggtgtcgtcccgtatcgatacgggacgacaccacactatgtcaagtaactccagaccacttcctcctccagcagaggtgtggtgaatgcattcaccacacctctgctggacggaaacaatccccgggtacgagaatcagggtgtggtgtcgtcccgtatcgatacgggacgacaccacaccctgattctcgtacccggggattgtttccgtccagcagaggtgtggtgaatgcattcaccacacctctgctggaaacctaccattaatgagacatgatgcggtggtgtggtgtcgtcccgtatcgatacgggacgacaccacaccaccgcatcatgtctcattaatggtaggtttccagcagaggtgtggtgaatg

SEQ ID NO: 21 - DNA, 980 bp, Artificial Sequence
  chemically synthesized
atccgtcacacctgctctggtggaatggactaagctagctagcgttttaaaaggtggtgttggctcccgtatatccgtcacacctgctctgtaaggggggggaatcgctttcgtcttaagatgacatggtgttggctcccgtatatccgtcacacctgctctgccggaccatccaatatcagctgtggtgttggctcccgtatatccgtcacacctgctctatccgtcacgcctgctctatccgtcacacctgctctggtgttggctcccgtatatccgtcacacctgctctatcaaatgtgcagatatcaagacgatttgtacaagatggtgttggctcccgtatatccgtcacacctgctctgtagatggcaaggcataagcgtccggaacgatagaatggtgttggctcccgtatatccgtcacacctgctctgtagatggcaaggcataagcgtccggaacgatagaatggtgttggctcccgtatatacgggagccaacaccaccttttaaaacgctagctagcttagtccattccaccagagcaggtgtgacggatatacgggagccaacaccatgtcatcttaagacgaaagcgattccccccccttacagagcaggtgtgacggatatacgggagccaacaccacagctgatattggatggtccggcagagcaggtgtgacggatatacgggagccaacaccagagcaggtgtgacggatagagcaggcgtgacggatagagcaggtgtgacggatatacgggagccaacaccatcttgtacaaatcgtcttgatatctgcacatttgatagagcaggtgtgacggatatacgggagccaacaccattctatcgttccggacgcttatgccttgccatctacagagcaggtgtgacggatatacgggagccaacaccattctatcgttccggacgcttatgccttgccatctacagagcaggtgtgacggat

SEQ ID NO: 22 - DNA, 1100 bp, Artificial Sequence
  chemically synthesized
atccgtcacacctgctctgccggaccatccaatatcagctgtggtgttggctcccgtatatccgtcacacctgctctggtggaatggactaagctagctagcgttttaaaaggtggtgttggctcccgtatatccgtcacacctgctcttaaagtagaggctgttctccagacgtcgcaggaggatggtgttggctcccgtatatccgtcacacctgctctgtagatggcaaggcataagcgtccggaacgatagaatggtgttggctcccgtatatccgtcacacctgctctgtagatggcaaggcataagcgtccggaacgatagaatggtgttggctcccgtatatacgggagccaacaccacagctgatattggatggtccggcagagcaggtgtgacggatatccgtcacacctgctcttgggcaggagcgagagactctaatggtaagcaagaatggtgttggctcccgtatatccgtcacacctgctctccaacaaggcgaccgaccgcatgcagatagccaggttggtgttggctcccgtatatacgggagccaacaccacagctgatattggatggtccggcagagcaggtgtgacggatatacgggagccaacaccaccttttaaaacgctagctagcttagtccattccaccagagcaggtgtgacggatatacgggagccaacaccatcctcctgcgacgtctggagaacagcctctactttaagagcaggtgtgacggatatacgggagccaacaccattctatcgttccggacgcttatgccttgccatctacagagcaggtgtgacggatatacgggagccaacaccattctatcgttccggacgcttatgccttgccatctacagagcaggtgtgacggatatccgtcacacctgctctgccggaccatccaatatcagctgtggtgttggctcccgtatatacgggagccaacaccattcttgcttaccattagagtctctcgctcctgcccaagagcaggtgtgacggatatacgggagccaacaccaacctggctatctgcatgcggtcggtcgccttgttggagagcaggtgtgacggat

SEQ ID NO: 23 - DNA, 1088 bp, Artificial Sequence
  chemically synthesized
atccgtcacacctgctctgtagatggcaaggcataagcgtccggaacgatagaatggtgttggctcccgtatatccgtcacacctgctctaaccaaaagggtaggagaccaagctagcgatttggatggtgttggctcccgtatatccgtcacacctgctctgccggaccatccaatatcagctgtggtgttggctcccgtatatccgtcacacctgctctgaagcctaacggagaagatggccctactgccgtaggtggtgttggctcccgtatatccgtcacacctgctctactaaacaagggcaaactgtaaacacagtaggggcgtggtgttggctcccgtatatccgtcacacctgctctggtgttggctcccgtatagcttggctcccgtatggtgttggctcccgtatatccgtcacacctgctctgtcgcgatgatgagcagcagcgcaggagggagggggtggtgttggctcccgtatatccgtcacacctgctctgatcagggaagacgccaacactggtgttggctcccgtatatacgggagccaacaccattctatcgttccggacgcttatgccttgccatctacagagcaggtgtgacggatatacgggagccaacaccatccaaatcgctagcttggtctcctacccttttggttagagcaggtgtgacggatatacgggagccaacaccacagctgatattggatggtccggcagagcaggtgtgacggatatacgggagccaacaccacctacggcagtagggccatcttctccgttaggcttcagagcaggtgtgacggatatacgggagccaacaccacgcccctactgtgtttacagtttgcccttgtttagtagagcaggtgtgacggatatacgggagccaacaccatacgggagccaagctatacgggagccaacaccagagcaggtgtgacggatatacgggagccaacaccaccccctccctcctgcgctgctgctcatcatcgcgacagagcaggtgtgacggatatacgggagccaacaccagtgttggcgtcttccctgatcagagcaggtgtgacggat

SEQ ID NO: 24 - DNA, 1070 bp, Artificial Sequence
  chemically synthesized
atccgtcacacctgctctgtccaaaggctacgcgttaacgtggtgttggctcccgtatatccgtcacacctgctctggagcaatatggtggagaaacgtggtgttggctcccgtatatccgtcacacctgctctgccggaccatccaatatcagctgtggtgttggctcccgtatatccgtcacacctgctctgaacaggatagggattagcgagtcaactaagcagcatggtgttggctcccgtatatccgtcacacctgctctggcggacaggaaataagaatgaacgcaaaatttatctggtgttggctcccgtatatccgtcacacctgctctacgcaacgcgacaggaacattcattatagaatgtgttggtgttggctcccgtatatccgtcacacctgctctcggctgcaatgcgggagagtaggggggaaccaaacctggtgttggctcccgtatatccgtcacacctgctctatgactggaacacgggtatcgatgattagatgtccttggtgttggctcccgtatatacgggagccaacaccacgttaacgcgtagcctttggacagagcaggtgtgacggatatacgggagccaacaccacgtttctccaccatattgctccagagcaggtgtgacggatatacgggagccaacaccacagctgatattggatggtccggcagagcaggtgtgacggatatacgggagccaacaccatgctgcttagttgactcgctaatccctatcctgttcagagcaggtgtgacggatatacgggagccaacaccagataaattttgcgttcattcttatttcctgtccgccagagcaggtgtgacggatatacgggagccaacaccaacacattctataatgaatgttcctgtcgcgttgcgtagagcaggtgtgacggatatacgggagccaacaccaggtttggttcccccctactctcccgcattgcagccgagagcaggtgtgacggatatacgggagccaacaccaaggacatctaatcatcgatacccgtgttccagtcatagagcaggtgtgacggat

(19) United States
(12) Patent Application Publication
(10) Pub. No.: US 2009/0186342 A1
(43) Pub. Date: Jul. 23, 2009

(54) Methods of producing competitive aptamer fret reagents and assays

(75) Inventors: John G. Bruno, San Antonio, TX (US); Joseph Chanpong, Chapel Hill, NC (US)

Correspondence Address: LOEFFLER JONAS & TUGGEY, LLP, 755 EAST MULBERRY STREET, SUITE 200, SAN ANTONIO, TX, 78212, US

(73) Assignee: PRONUCLEOTEIN BIOTECHNOLOGIES, LLC

(21) Appl. No.: 12/011,675

(22) Filed: Jan. 29, 2008

Related U.S. Application Data
(63) Continuation-in-part of application No. 11/433,283, filed on May 12, 2006.

(51) Int. Cl. C12Q 1/68

(52) U.S. Cl. 435/6

(57) ABSTRACT

Methods are described for the production and use of fluorescence resonance energy transfer (FRET)-based competitive displacement aptamer assay formats. The assay schemes involve FRET in which the analyte (target) is quencher (Q)-labeled and previously bound by a fluorophore (F)-labeled aptamer such that when unlabeled analyte is added to the system and excited by specific wavelengths of light, the fluorescence intensity of the system changes in proportion to the amount of unlabeled analyte added. Alternatively, the aptamer can be Q-labeled and previously bound to an F-labeled analyte so that when unlabeled analyte enters the system, the fluorescence intensity also changes in proportion to the amount of unlabeled analyte. The F or Q is covalently linked to nucleotide triphosphates (NTPs), which are incorporated into the aptamer by various nucleic acid polymerases, such as Taq or Deep Vent Exo⁻ during PCR or asymmetric PCR, and then selected by affinity chromatography, size-exclusion, and fluorescence techniques.

### CROSS-REFERENCE TO RELATED APPLICATIONS

This application is a continuation-in-part of copending U.S. application Ser. No. 11/433,283 filed on May 12, 2006, which is incorporated herein by reference.

### BACKGROUND OF THE INVENTION

1. Field of the Invention

The present invention relates to the field of aptamer- and nucleic acid-based diagnostics. More particularly, it relates to methods for the production and use of fluorescence resonance energy transfer (“FRET”) DNA or RNA aptamers for competitive displacement aptamer assay formats. The present invention provides for aptamer-related FRET assay schemes involving competitive displacement formats in which the aptamer contains fluorophores (“F”) (is F-labeled) and the target contains quenchers (“Q”) (is Q-labeled), or vice versa. The aptamer can be F-labeled or Q-labeled by incorporation of the F or Q derivatives of nucleotide triphosphates. Incorporation may be accomplished by simple chemical conjugations through bifunctional linkers, or key functional groups such as aldehydes, carbodiimides, carboxyls, N-hydroxy-succinimide (NHS) esters, thiols, etc.

2. Background Information

Competitive displacement aptamer FRET is a new class of assay desirable for its use in rapid (within minutes), one-step, homogeneous assays involving no wash steps (simple bind and detect quantitative assays). Others have described FRET-aptamer methods for various target analytes that consist of placing the F and Q moieties either on the 5′ and 3′ ends respectively to act like a “molecular (aptamer) beacon” or placing only F in the heart of the aptamer structure to be “quenched” by another proximal F or the DNA or RNA itself. These preceding FRET-aptamer methods are all highly engineered and based on some prior knowledge of particular aptamer sequences and secondary structures, thereby enabling clues as to where F might be placed in order to optimize FRET results.

### SUMMARY OF THE INVENTION

The nucleic acid-based “molecular beacons” snap open upon binding to an analyte or upon hybridizing to a complementary sequence, but beacons have always been end-labeled with F and Q at the 3′ and 5′ ends. The present invention provides that F-labeled or Q-labeled aptamers may be labeled anywhere in their structure that places the F or Q within the Förster distance of approximately 60-85 Angstroms of the corresponding F or Q on the labeled target analyte to achieve quenching prior to or after target analyte binding to the aptamer “binding pocket” (typically a “loop” in the secondary structure). The F and Q molecules used can include any number of appropriate fluorophores and quenchers as long as they are spectrally matched so the emission spectrum of F overlaps significantly (almost completely) with the absorption spectrum of Q.

A process in which F and Q are incorporated into an aptamer population is generally referred to as “doping.” The present invention provides a new method for natural selection of F-labeled or Q-labeled aptamers that contain F-NTPs or Q-NTPs in the heart of an aptamer binding loop or pocket by PCR, asymmetric PCR (using a 100:1 forward:reverse primer ratio), or other enzymatic means. The present invention describes a strain of aptamer in which F and Q are incorporated into an aptamer population via their nucleotide triphosphate derivatives (for example, Alexfluor™-NTP's, Cascade Blue®-NTP's, Chromatide®-NTP's, fluorescein-NTP's, rhodamine-NTP's, Rhodamine Green™-NTP's, tetramethylrhodamine-dNTP's, Oregon Green®-NTP's, and Texas Red®-NTP's may be used to provide the fluorophores, while dabcyl-NTP's, Black Hole Quencher or BHQ™-NTP's, and QSY™ dye-NTP's may be used for the quenchers) by PCR after several rounds of selection and amplification without the F- and Q-modified bases. The advantage of this F or Q “doping” method is two-fold: 1) the method allows nature to take its course and select the most sensitive F-labeled or Q-labeled aptamer target interactions in solution, and 2) the positions of F or Q within the aptamer structure can be determined via exonuclease digestion of the F-labeled or Q-labeled aptamer followed by mass spectral analysis of the resulting fragments, thereby eliminating the need to “engineer” the F or Q moieties into a prospective aptamer binding pocket or loop. Sequence and mass spectral data can be used to further optimize the competitive aptamer FRET assay performance after natural selection as well.

If the target molecule is a larger water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTP's that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule (generally defined as a molecule with molecular weight of ≦1,000 Daltons), then exposure of the nascent F-labeled and Q-labeled DNA or RNA random library to the target is done by immobilizing the target. The small molecule can be immobilized on a column, membrane, plastic or glass bead, magnetic bead, quantum dot, or other matrix. If no functional group is available on the small molecule for immobilization, the target can be immobilized by the Mannich reaction (formaldehyde-based condensation reaction) on a PharmaLink™ column from Pierce Chemical Co. Elution of bound DNA from the small molecule affinity column, membrane, beads or other matrix by use of 0.2-3.0M sodium acetate at a pH of between 3 and 7.

The candidate FRET-aptamers are separated based on physical properties such as charge or weak interactions by various types of HPLC, digested at each end with specific exonucleases (snake venom phosphodiesterase on the 3′ end and calf spleen phosphodiesterase on the 5′ end). The resulting oligonucleotide fragments, each one bases shorter than the predecessor, are subjected to mass spectral analysis which can reveal the nucleotide sequences as well as the positions of F and Q within the FRET-aptamers. Once the FRET-aptamer sequence is known with the positions of F and Q, it can be further manipulated during solid-phase DNA or RNA synthesis in an attempt to make the FRET assay more sensitive and specific.

The competitive displacement aptamer FRET assay format of the present invention is unique. The competitive format generally requires a lower affinity aptamer in order to be able to release the F-labeled or Q-labeled target analyte and allow competition for the binding site. This may lead to less sensitivity in some assays.

When running an assay, an aptamer is incorporated. In order to interact with the target molecule, the aptamer has a binding pocket or site. It is anticipated in some embodiments that the binding pocket is comprised of 3 to 6 nucleotides. These 3 or more nucleotides have a specific sequence or arrangement so as to confer the appropriate volume and conformation in 3-dimensional space to enable optimal binding to the target molecule. Where the target molecule can be any of the type described herein.

The described competitive FRET aptamer uses unique aptamer sequences. The following sequences are all aptamers that bind foodborne pathogens such as E. coli O157:H7, Salmonella typhimurium and a surface protein from Listeria monocytogenes called “Listeriolysin.” F=forward and R=reverse primed sequences. The invention described herein may use one or more of the following aptamer sequences (the following aptamer sequences are collectively referred to as the “SEQ Aptamers.”) (The SEQ Aptamer identifiers are arranged alphabetically by aptamer target, and are listed 5′ to 3′ from left to right.):

### Acetylcholine (ACh) Aptamer Sequences:

ACh1a For
ATACGGGAGCCAACACCACGATACCCGCTTATGAATTTTAAATTAATTGT
GATCAGAGCAGGTGTGACGGAT
ACh1a Rev
ATCCGTCACACCTGCTCTGATCACAATTAATTTAAAATTCATAAGCGGGT
ATCGTGGTGTTGGCTCCCGTAT
ACh 1b For
ATACGGGAGCCAACACCAACTTTCACACATACTTGTTATACCACACGATC
TTTTAGAGCAGGTGTGACGGAT
ACh 1b Rev
ATCCGTCACACCTGCTCTAAAAGATCGTGTGGTATAACAAGTATGTGTGA
AAGTTGGTGTTGGCTCCCGTAT
ACh 2 For
ATACGGGAGCCAACACCACTTTGTAACTCATTTGTAGTTTGGGTTGCTCC
CCCTAGAGCAGGTGTGACGGAT
ACh 2 Rev
ATCCGTCACACCTGCTCTAGGGGGAGCAACCCAAACTACAAATGAGTTAC
AAAGTGGTGTTGGCTCCCGTAT
ACh 3 For
ATACGGGAGCCAACACCATTTCCCGCTTATCTTCATCCACTGCTTAGCAT
ATGTAGAGCAGGTGTGACGGAT
ACh 3 Rev
ATCCGTCACACCTGCTCTACATATGCTAAGCAGTGGATGAAGATAAGCGG
GAAATGGTGTTGGCTCCCGTAT
ACh 5 For
ATACGGGAGCCAACACCAGGCACTGTATCACACCGTCAAGAATGTGATCC
CCTGAGAGCAGGTGTGACGGAT
ACh 5 Rev
ATCCGTCACACCTGCTCTCAGGGGATCACATTCTTGACGGTGTGATACAG
TGCCTGGTGTTGGCTCCCGTAT
ACh 6 For
ATACGGGAGCCAACACCATGTCATTTACCTTCATCATGACAGTGTTAGTA
TACGAGAGCAGGTGTGACGGAT
ACh 6Rev
ATCCGTCACACCTGCTCTAGGGGATCAAAGCTATGCGACCATGCGAGTGG
ATACTGGTGTTGGCTCCCGTAT
ACh 7 For
ATACGGGAGCCAACACCAGTTGCCGCCTACCTTGATTATTCTACATTACC
CGTTAGAGCAGGTGTGACGGAT
ACh 7 Rev
ATCCGTCACACCTGCTCTAACGGGTAATGTAGAATAATCAAGGTAGGCGG
CAACTGGTGTTGGCTCCCGTAT
ACh 8 For
ATACGGGAGCCAACACCAGTATACATACGAAGAGTTGAAACCAATGCTTC
GTTCAGAGCAGGTGTGACGGAT
ACh 8 Rev
ATCCGTCACACCTGCTCTGAACGAAGCATTGGTTTCAACTCTTCGTATGT
ATACTGGTGTTGGCTCCCGTAT
ACh 9 For
ATACGGGAGCCAACACCATACCCCGAATGGCTGTTTTCAGTACCAATATG
ACTCAGAGCAGGTGTGACGGAT
ACh 9 Rev
ATCCGTCACACCTGCTCTGAGTCATATTGGTACTGAAAACAGCCATTCGG
GGTATGGTGTTGGCTCCCGTAT
ACh 10 For
ATACGGGAGCCAACACCACTGTCACGATCGTCGTTCCTTTTAATCCTTGT
GTCTAGAGCAGGTGTGACGGAT
ACh 10 Rev
ATCCGTCACACCTGCTCTAGACACAAGGATTAAAAGGAACGACGATCGTG
ACAGTGGTGTTGGCTCCCGTAT
ACh 11 For
ATACGGGAGCCAACACCACTGGACACTGACCCTCGCACTAGCTTTCTGAC
GGGTAGAGCAGGTGTGACGGAT
ACh 11 Rev
ATCCGTCACACCTGCTCTACCCGGCCGAAGAATAGTGCTCGGTACTTAGT
CGCGTGGTGTTGGCTCCCGTAT
ACh 12 For
ATACGGGAGCCAACACCATTTGGACTTTAAATAGTGGACTCCTTCTTTGT
CTCGAGAGCAGGTGTGACGGAT
ACh 12 Rev
ATCCGTCACACCTGCTCTCGAGACAAAGAAGGAGTCCACTATTTAAAGTC
CAAATGGTGTTGGCTCCCGTAT
A25 For
ATACGGGAGCCAACACCA-TCATTTGCAAATATGAATTCCACTTAAAGAA
ATTCA-AGAGCAGGTGTGACGGAT
A25 Rev
ATCCGTCACACCTGCTCTTGAATTTCTTTAAGTGGAATTCATATTTGCAA
ATGATGGTGTTGGCTCCCGTAT

### Acyl Homoserine Lactone (AHL) Quorum Sensing Molecules (N-Decanoyl-DL-Homoserine Lactone)

Dec AHL 1For
ATACGGGAGCCAACACCATCCTAACTGGTCTAATTTTTGCTGTTACCGAT
CCCGAGAGCAGGTGTGACGGAT
Dec AHL 1 Rev
ATCCGTCACTCCTGCTCTCGGGATCGGTAACAGCAAAAATTAGACCAGTT
AGGATGGTGTTGGCTCCCGTAT
Dec AHL 13 For
ATACGGGAGCCAACACCAGCCTGACGAAAAAATTTTATCACTAAGTGATA
CGCAAGAGCAGGTGTGACGGAT
Dec AHL 13 Rev
ATCCGTCACACCTGCTCTTGCGTATCACTTAGTGATAAAATTTTTTCGTC
AGGCTGGTGTTGGCTCCCGTAT
Dec AHL 14 For
ATACGGGAGCCAACACCAGACCTACTTCAGAAACGGAAATGTTCTTAGCC
GTCAGAGCAGGTGTGACGGAT
Dec AHL 14 Rev
ATCCGTCACACCTGCTCTGACGGCTAAGAACATTTCCGTTTCTGAAGTAG
GTCTGGTGTTGGCTCCCGTAT
Dec AHL 15 For
ATACGGGAGCCAACACCAGGCCAACGAAACTCCTACTACATATAATGCTT
ATGCAGAGCAGGTGTGACGGAT
Dec AHL 15 Rev
ATCCGTCACACCTGCTCTGCATAAGCATTATATGTAGTAGGAGTTTCGTT
GGCCTGGTGTTGGCTCCCGTAT
Dec AHL 17 For
ATACGGGAGCCAACACCATCCTAACTGGTCTAATTTTTGCTGTTACCGAT
CCCGAGAGCAGGTGTGACGGAT
Dec AHL 17 Rev
ATCCGTCACACCTGCTCTCGGGATCGGTAACAGCAAAAATTAGACCAGTT
AGGATGGTGTTGGCTCCCGTAT

Bacillus thuringiensis Spore Aptamer Sequence:

CATCCGTCACACCTGCTCTGGCCACTAACATGGGGACCAGGTGGTGTTGG
CTCCCGTATC

### Botulinum Toxin (BoNT Type A) Aptamer Sequences:

### BoNT A Holotoxin (Heavy Chain Plus Light Chain Linked Together)

CATCCGTCACACCTGCTCTGCTATCACATGCCTGCTGAAGTGGTGTTGGC
TCCCGTATCA

### BoNT A 50 kd Enzymatic Light Chain

BoNT A Light Chain 1
CATCCGTCACACCTGCTCTGGGGATGTGTGGTGTTGGCTCCCGTATCAAG
GGCGAATTCT
BoNT A Light Chain 2
CATCCGTCACACCTGCTCTGATCAGGGAAGACGCCAACACGTGGTGTTGG
CTCCCGTATCA
BoNT A Light Chain 3
CATCCGTCACACCTGCTCTGGGTGGTGTTGGCTCCCGTATCAAGGGCGAA
TTCTGCAGATA

Campylobacter jejuni Binding Aptamers:

C1
CATCCGTCACACCTGCTCTGGGGAGGGTGGCGCCCGTCTCGGTGGTGTTG
GCTCCCGTATCA
C2
CATCCGTCACACCTGCTCTGGGATAGGGTCTCGTGCTAGATGTGGTGTTG
GCTCCCGTATCA
C3
CATCCGTCACACCTGCTCTGGACCGGCGCTTATTCCTGCTTGTGGTGTTG
GCTCCCGTATCA
C4
CATCCGTCACACCTGCYCTGGAGCTGATATTGGATGGTCCGGTGGTGTTG
GCTCCCGTATCA
C5
CATCCGTCACACCTGCYCYGCCCAGAGCAGGTGTGACGGATGTGGTGTTG
GCTCCCGTATCA
C6
CATCCGTCACACCTGCYCYGCCGGACCATCCAATATCAGCTGTGGTGTTG
GCTCCCGTATCA

### Diazinon Binding Aptamers

D12 Forward
ATACGGGAGCCAACACCATTAAATCAATTGTGCCGTGTTGGTCTTGTCTC
ATCGAGAGCAGGTGTGACGGAT
D12 Reverse
ATCCGTCACACCTGCTCTCGATGAGACAAGACCAACACGGCACAATTGAT
TTAATGGTGTTGGCTCCCGTAT
D17 Forward
ATACGGGAGCCAACACCATTTTTATTATCGGTATGATCCTACGAGTTCCT
CCCAAGAGCAGGTGTGACGGAT
D17 Reverse
ATCCGTCACACCTGCTCTTGGGAGGAACTCGTAGGATCATACCGATAATA
AAAATGGTGTTGGCTCCCGTAT
D18 Forward
ATACGGGAGCCAACACCACCGTATATCTTATTATGCACAGCATCACGAAA
GTGCAGAGCAGGTGTGACGGAT
D18 Reverse
ATCCGTCACACCTGCTCTGCACTTTCGTGATGCTGTGCATAATAAGATAT
ACGGTGGTGTTGGCTCCCGTAT
D19 Forward
ATACGGGAGCCAACACCATTAACGTTAAGCGGCCTCACTTCTTTTAATCC
TTTCAGAGCAGGTGTGACGGAT
D19 Reverse
ATCCGTCACACCTGCTCTGAAAGGATTAAAAGAAGTGAGGCCGCTTAACG
TTAATGGTGTTGGCTCCCGTAT
D20 Forward
ATCCGTCACACCTGCTCTAATATAGAGGTATTGCTCTTGGACAAGGTACA
GGGATGGTGTTGGCTCCCGTAT
D20 Reverse
ATACGGGAGCCAACACCATCCCTGTACCTTGTCCAAGAGCAATACCTCTA
TATTAGAGCAGGTGTGACGGAT
D25 Forward
ATACGGGAGCCAACACCATTAACGTTAAGCGGCCTCACTTCTTTTAATCC
TTTCAGAGCAGGTGTGACGGAT
D25 Reverse
ATCCGTCACACCTGCTCTGAAAGGATTAAAAGAAGTGAGGCCGCTTAACG
TTAATGGTGTTGGCTCCCGTAT

Glucosamine (from LPS) Forward Aptamer Sequences:

G 1 For
ATCCGTCACACCTGCTCTAATTAGGATACGGGGCAACAGAACGAGAGGGG
GGAATGGTGTTGGCTCCCGTAT
G 2 For
ATCCGTCACACCTGCTCTCGGACCAGGTCAGACAAGCACATCGGATATCC
GGCTGGTGTTGGCTCCCGTAT
G 4 For
ATCCGTCACACCTGCTCTAATTAGGATACGGGGCAACAGAACGAGAGGGG
GGAATGGTGTTGGCTCCCGTAT
G 5 For
ATCCGTCACACCTGCTCTTGAGTCAAAGAGTTTAGGGAGGAGCTAACATA
ACAGTGGTGTTGGCTCCCGTAT
G 7 For
ATCCGTCACACCTGCTCTAACAACAATGCATCAGCGGGCTGGGAACGCAT
GCGGTGGTGTTGGCTCCCGTAT
G 8 For
ATCCGTCACACCTGCTCTGAACAGGTTATAAGCAGGAGTGATAGTTTCAG
GATCTGGTGTTGGCTCCCGTAT
G 9 For
ATCCGTCACACCTGCTCTCGGCGGCTCGCAAACCGAGTGGTCAGCACCCG
GGTTGGTGTTGGCTCCCGTAT
G 10 For
ATCCGTCACACCTGCTCTGCGCAAGACGTAATCCACAAGACCGTGAAAAC
ATAGTGGTGTTGGCTCCCGTAT

Glucosamine (from LPS) Reverse Sequences:

G 1 Rev
ATACGGGAGCCAACACCATTCCCCCCTCTCGTTCTGTTGCCCCGTATCCT
AATTAGAGCAGGTGTGACGGAT
G 2 Rev
ATACGGGAGCCAACACCAGCCGGATATCCGATGTGCTTGTCTGACCTGGT
CCGAGAGCAGGTGTGACGGAT
G 4 Rev
ATACGGGAGCCAACACCATTCCCCCCTCTCGTTCTGTTGCCCCGTATCCT
AATTAGAGCAGGTGTGACGGAT
G 5 Rev
ATACGGGAGCCAACACCACTGTTATGTTAGCTCCTCCCTAAACTCTTTGA
CTCAAGAGCAGGTGTGACGGAT
G 7 Rev
ATACGGGAGCCAACACCACCGCATGCGTTCCCAGCCCGCTGATGCATTGT
TGTTAGAGCAGGTGTGACGGAT
G 8 Rev
ATACGGGAGCCAACACCAGATCCTGAAACTATCACTCCTGCTTATAACCT
GTTCAGAGCAGGTGTGACGGAT
G 9 Rev
ATACGGGAGCCAACACCAACCCGGGTGCTGACCACTCGGTTTGCGAGCCG
CCGAGAGCAGGTGTGACGGAT
G 10 Rev
ATACGGGAGCCAACACCACTATGTTTTCACGGTCTTGTGGATTACGTCTT
GCGCAGAGCAGGTGTGACGGAT

KDO Antigen from LPS (Forward Primed):

K 2 For
ATCCGTCACACCTGCTCTAGGCGTAGTGACTAAGTCGCGCGAAAATCACA
GCATTGGTGTTGGCTCCCGTAT
K 5 For
ATCCGTCACACCTGCTCTCAGCGGCAGCTATACAGTGAGAACGGACTAGT
GCGTTGGTGTTGGCTCCCGTAT
K 7 For
ATCCGTCACACCTGCTCTGGCAAATAATACTAGCGATGATGGATCTGGAT
AGACTGGTGTTGGCTCCCGTAT
K 8 For
ATCCGTCACACCTGCTCTGGGGGTGCGACTTAGGGTAAGTGGGAAAGACG
ATGCTGGTGTTGGCTCCCGTAT
K 9 For
ATCCGTCACACCTGCTCTCAAGAGGAGATGAACCAATCTTAGTCCGACAG
GCGGTGGTGTTGGCTCCCGTAT
K 10 For
ATCCGTCACACCTGCTCTGGCCCGGAATTGTCATGACGTCACCTACACCT
CCTGTGGTGTTGGCTCCCGTAT

KDO Antigen from LPS (Reverse Primed):

K 2 Rev
ATACGGGAGCCAACACCAATGCTGTGATTTTCGCGCGACTTAGTCACTAC
GCCTAGAGCAGGTGTGACGGAT
K 5 Rev
ATACGGGAGCCAACACCAACGCACTAGTCCGTTCTCACTGTATAGCTGCC
GCTGAGAGCAGGTGTGACGGAT
K 7 Rev
ATACGGGAGCCAACACCAGTCTATCCAGATCCATCATCGCTAGTATTATT
TGCCAGAGCAGGTGTGACGGAT
K 8 Rev
ATACGGGAGCCAACACCAGCATCGTCTTTCCCACTTACCCTAAGTCGCAC
CCCCAGAGCAGGTGTGACGGAT
K 9 Rev
ATACGGGAGCCAACACCACCGCCTGTCGGACTAAGATTGGTTCATCTCCT
CTTGAGAGCAGGTGTGACGGAT
K 10 Rev
ATACGGGAGCCAACACCACAGGAGGTGTAGGTGACGTCATGACAATTCCG
GGCCAGAGCAGGTGTGACGGAT

Leishmania donovani Binding Aptamer Sequences:
Leishmania donovani Clone: 940-3

Forward:
GATACGGGAGCCAACACCACCCGTATCGTTCCCAATGCACTCAGAGCAGG
TGTGACGGATG
Reverse:
CATCCGTCACACCTGCTCTGAGTGCATTGGGAACGATACGGGTGGTGTTG
GCTCCCGTATG

Leishmania donovani Clone: 940-5

Forward:
GATACGGGAGCCAACACCACGTTCCCATACAAGTTACTGACAGAGCAGGT
GTGACGGATG
Reverse:
CATCCGTCACACCTGCTCTGTCAGTAACTTGTATGGGAACGTGGTGTTGG
CTCCCGTATC

Whole LPS from E. coli O111:B4 Binding Aptamer Sequences (Forward Primed):

LPS 1 For
ATCCGTCACCCCTGCTCTCGTCGCTATGAAGTAACAAAGATAGGAGCAAT
CGGGTGGTGTTGGCTCCCGTAT
LPS 3 For
ATCCGTCACACCTGCTCTAACGAAGACTGAAACCAAAGCAGTGACAGTGC
TGAATGGTGTTGGCTCCCGTAT
LPS 4 For
ATCCGTCACACCTGCTCTCGGTGACAATAGCTCGATCAGCCCAAAGTCGT
CAGATGGTGTTGGCTCCCGTAT
LPS 6 For
ATCCGTCACACCTGCTCTAACGAAATAGACCACAAATCGATACTTTATGT
TATTGGTGTTGGCTCCCGTAT
LPS 7 For
ATCCGTCACACCTGCTCTGTCGAATGCTCTGCCTGGAAGAGTTGTTAGCA
GGGATGGTGTTGGCTCCCGTAT
LPS 8 For
ATCCGTCACACCTGCTCTTAAGCCGAGGGGTAAATCTAGGACAGGGGTCC
ATGATGGTGTTGGCTCCCGTAT
LPS 9 For
ATCCGTCACACCTGCTCTACTGGCCGGCTCAGCATGACTAAGAAGGAAGT
TATGTGGTGTTGGCTCCCGTAT
LPS 10 For
ATCCGTCACACCTGCTCTGGTACGAATCACAGGGGATGCTGGAAGCTTGG
CTCTTGGTGTTGGCTCCCGTAT

Whole LPS from E. coli O111:B4 Binding Aptamer Sequences (Reverse Primed):

LPS 1 Rev
ATACGGGAGCCAACACCACCCGATTGCTCCTATCTTTGTTACTTCATAGC
GACGAGAGCAGGGGTGACGGAT
LPS 3 Rev
ATACGGGAGCCAACACCATTCAGCACTGTCACTGCTTTGGTTTCAGTCTT
CGTTAGAGCAGGTGTGACGGAT
LPS 4 Rev
ATACGGGAGCCAACACCATCTGACGACTTTGGGCTGATCGAGCTATTGTC
ACCGAGAGCAGGTGTGACGGAT
LPS 6 Rev
ATACGGGAGCCAACACCAATAACATAAAGTATCGATTTGTGGTCTATTTC
GTTAGAGCAGGTGTGACGGAT
LPS 7 Rev
ATACGGGAGCCAACACCATCCCTGCTAACAACTCTTCCAGGCAGAGCATT
CGACAGAGCAGGTGTGACGGAT
LPS 8 Rev
ATACGGGAGCCAACACCATCATGGACCCCTGTCCTAGATTTACCCCTCGG
CTTAAGAGCAGGTGTGACGGAT
LPS 9 Rev
ATACGGGAGCCAACACCACATAACTTCCTTCTTAGTCATGCTGAGCCGGC
CAGTAGAGCAGGTGTGACGGAT
LPS 10 Rev
ATACGGGAGCCAACACCAAGAGCCAAGCTTCCAGCATCCCCTGTGATTCG
TACCAGAGCAGGTGTGACGGAT

### Methylphosphonic Acid (MPA) Binding Aptamer Sequences:

MPA Forward
ATACGGGAGCCAACACCATTAAATCAATTGTGCCGTGTTCCTCTTGTCTC
ATCGAGAGCAGGTTGTACGGAT
MPA Reverse
ATCCGTACAACCTGCTCTCGATGAGACAAGAGGAACACGGCACAATTGAT
TTAATGGTGTTGGCTCCCGTAT

### Malathion Binding Aptamer Sequences:

M17 Forward
ATACGGGAGCCAACACCAGCAGTCAAGAAGTTAAGAGAAAAACAATTGTG
TATAAGAGCAGGTGTGACGGAT
M17 Reverse
ATCCGTCACACCTGCTCTTATACACAATTGTTTTTCTCTTAACTTCTTGA
CTGCTGGTGTTGGCTCCCGTAT
M21 Forward
ATCCGTCACACCTGCTCTGCGCCACAAGATTGCGGAAAGACACCCGGGGG
GCTTGGTGTTGGCTCCCGTAT
M21 Reverse
ATACGGGAGCCAACACCAAGCCCCCCGGGTGTCTTTCCGCAATCTTGTGG
CGCAGAGCAGGTGTGACGGAT
M25 Forward
ATCCGTCACACCTGCTCTGGCCTTATGTAAAGCGTTGGGTGGTGTTGGCT
CCCGTAT
M25 Reverse
ATACGGGAGCCAACACCACCCAACGCTTTACATAAGGCCAGAGCAGGTGT
GACGGAT

### Poly-D-Glutamic Acid Binding Aptamer Sequences:

PDGA 2F
CATCCGTCACACCTGCTCTGGTTCGCCCCGGTCAAGGAGAGTGGTGTTGG
CTCCCGTATC
PDGA 2R
GATACGGGAGCCAACACCACTCTCCTTGACCGGGGCGAACCAGAGCAGGT
GTGACGGATG
PDGA 5F
CATCCGTCACACCTGCTCTGGATAAGATCAGCAACAAGTTAGTGGTGTTG
GCTCCCGTATC
PDGA 5R
GATACGGGAGCCAACACCACTAACTTGTTGCTGATCTTATCAGAGCAGGT
GTGACGGATG

### Rough Ra Mutant LPS Core Antigen Binding Aptamer Sequences (Forward Primed):

R 1F
ATCCGTCACACCTGCTCTCCGCACGTAGGACCACTTTGGTACACGCTCCC
GTAGTGGTGTTGGCTCCCGTAT
R 5F
ATCCGTCACACCTGCTCTACGGATGAACGAAGATTTTAAAGTCAAGCTAA
TGCATGGTGTTGGCTCCCGTAT
R 6F
ATCCGTCACACCTGCTCTGTAGTGAAGAGTCCGCAGTCCACGCTGTTCAA
CTCATGGTGTTGGCTCCCGTAT
R 7F
ATCCGTCACACCTGCTCTACCGGCTGGCACGGTTATGTGTGACGGGCGAA
GATATGGTGTTGGCTCCCGTAT
R 8F
ATCCGTCACACCTGCTCTACCGGCTGGCACGGTTATGTGTGACGGGCGAA
GATATGGTGTTGGCTCCCGTAT
R 9F
ATCCGTCACACCTGCTCTGCGTGTGGAGCGCCTAGGTGAGTGGTGTTGGC
TCCCGTAT
R 10F
ATCCGTCACACCTGCTCTGATGTCCCTTTGAAGAGTTCCATGACGCTGGC
TCCTTGGTGTTGGCTCCCGTAT

### Roueh Ra Mutant LPS Core Antigen Binding Aptamer Sequences (Reverse Primed):

R 1R
ATACGGGAGCCAACACCACTACGGGAGCGTGTACCAAAGTGGTCCTACGT
GCGGAGAGCAGGTGTGACGGAT
R 5R
ATACGGGAGCCAACACCATGCATTAGCTTGACTTTAAAATCTTCGTTCAT
CCGTAGAGCAGGTGTGACGGAT
R 6R
ATACGGGAGCCAACACCATGAGTTGAACAGCGTGGACTGCGGACTCTTCA
CTACAGAGCAGGTGTGACGGAT
R 7R
ATACGGGAGCCAACACCATATCTTCGCCCGTCACACATAACCGTGCCAGC
CGGTAGAGCAGGTGTGACGGAT
R 8R
ATACGGGAGCCAACACCATATCTTCGCCCGTCACACATAACCGTGCCAGC
CGGTAGAGCAGGTGTGACGGAT
R 9R
ATACGGGAGCCAACACCACTCACCTAGGCGCTCCACACGCAGAGCAGGTG
TGACGGAT
R 10R
ATACGGGAGCCAACACCAAGGAGCCAGCGTCATGGAACTCTTCAAAGGGA
CATCAGAGCAGGTGTGACGGAT

### Soman Binding Aptamer Sequences:

Soman 20F
ATACGGGAGCCAACACCATAGTGTTGGGCCAATACGGTAACGTGTCCTTG
GAGAGCAGGTGTGACGGAT
Soman 20R
ATCCGTCACACCTGCTCTCCAAGGACACGTTACCGACGAATTGGCCCAAC
ACTATGGTGTTGGCTCCCGTAT
Soman 23F
ATACGGGAGCCAACACCACACATACGAGTTATCTCGAGTAGAGCATGTTT
TGCCAGAGCAGGTGTGACGGAT
Soman 23R
ATCCGTCACACCTGCTCTGGCAAAACATGCTCTACTCGAGATAACTCGTA
TGTGTGGTGTTGGCTCCCGTAT
Soman 24F
ATACGGGAGCCAACACCAGGCCATCTATTGTTCGTTTTTCTATTTATCTC
ACCCAGAGCAGGTGTGACGGAT
Somna 24R
ATCCGTCACACCTGCTCTGGGTGAGATAAATAGAAAAACGAACAATAGAT
GGCCTGGTGTTGGCTCCCGTAT
Soman 25F
ATACGGGAGCCAACACCACACATACGAGTTATCTCGAGTAGAGCATGTTT
TGCCAGAGCAGGTGTGACGGAT
Soman 25R
ATCCGTCACACCTGCTCTGGCAAAACATGCTCTACTCGAGATAACTCGTA
TGTGTGGTGTTGGCTCCCGTAT
Soman 28F
ATACGGGAGCCAACACCATCCATAGCTCATCTATACCCTCTTCCGAGTCC
CACCAGAGCAGGTGTGACGGAT
Soman 28R
ATCCGTCACACCTGCTCTGGTGGGACTCGGAAGAGGGTATAGATGAGCTA
TGGATGGTGTTGGCTCCCGTAT
Soman 33F
ATACGGGAGCCAACACCAGAGCAGGTGTGACGGATAGTGACGGATGCAGA
GCAGGTGTGACGGAT
Soman 33R
ATCCGTCACACCTGCTCTGCATCCGTCACTATCCGTCACACCTGCTCTGG
TGTTGGCTCCCGTAT
Soman 41F
ATACGGGAGCCAACACCACCTTATGACGCCTCAGTACCACATCGTTTAGT
CTGTAGAGCAGGTGTGACGGAT
Soman 41R
ATCCGTCACACCTGCTCTACAGACTAAACGATGTGGTACTGAGGCGTCAT
AAGGTGGTGTTGGCTCCCGTAT
Soman 45F
ATACGGGAGCCAACACCACCCGTTTTTGATCTAATGAGGATACAATATTC
GTCTAGAGCAGGTGTGACGGAT
Soman 45R
ATCCGTCACACCTGCTCTAGACGAATATTGTATCCTCATTAGATCAAAAA
CGGGTGGTGTTGGCTCCCGTAT
Soman 46F
ATACGGGAGCCAACACCATCGAGCTCCTTGGCCCCGTTAGGATTAACGTG
ATGTAGAGCAGGTGTGACGGAT
Soman 46R
ATCCGTCACACCTGCTCTACATCACGTTAATCCTAACGGGGCCAAGGAGC
TCGATGGTGTTGGCTCCCGTAT
Soman 47F
ATACGGGAGCCAACACCATCAGAACCAAATATACATCTTCCTATGATATG
GTGGAGAGCAGGTGTGACGGAT
Soman 47R
ATCCGTCACACCTGCTCTCCACCATATCATAGGAAGATGTATATTTGGTT
CTGATGGTGTTGGCTCCCGTAT
Soman 48F
ATACGGGAGCCAACACCACACGATTGCTCCTCTCATTGTTACTTCATAGC
GACGAGAGCAGGTGTGACGGAT
Soman 48R
ATCCGTCACACCTGCTCTCGTCGCTATGAAGTAACAATGAGAGGAGCAAT
CGTGTGGTGTTGGCTCCCGTAT

### Teichoic Acid or Lipoteichoic Acid Binding Aptamer Sequences:

T5 F
GATACGGGACGACACCACACTATGGGTCGTTTAGCATCAAGGCTAGCCAA
GCCAGCAGAGGTGTGGTGAATG
T5 R
CATTCACCACACCTCTGCTGGCTTGGCTAGCCTTGATGCTAAACGACCCA
TAGTGTGGTGTCGTCCCGTATC
T6 F
CATTCACCACACCTCTGCTGGAGGAGGAAGTGGTCTGGAGTTACTTGACA
TAGTGTGGTGTCGTCCCGTATC
T6 R
GATACGGGACGACACCACACTATGTCAAGTAACTCCAGACCACTTCCTCC
TCCAGCAGAGGTGTGGTGAATG
T7 F
CATTCACCACACCTCTGCTGGACGGAAACAATCCCCGGGTACGAGAATCA
GGGTGTGGTGTCGTCCCGTATC
T7 R
GATACGGGACGACACCACACCCTGATTCTCGTACCCGGGGATTGTTTCCG
TCCAGCAGAGGTGTGGTGAATG
T9 F
CATTCACCACACCTCTGCTGGAAACCTACCATTAATGAGACATGATGCGG
TGGTGTGGTGTCGTCCCGTATC
T9 R
GATACGGGACGACACCACACCACCGCATCATGTCTCATTAATGGTAGGTT
TCCAGCAGAGGTGTGGTGAATG

E. coli O157 Lipopolysaccharide (LPS)

E-5F
ATCCGTCACACCTGCTCTGGTGGAATGGACTAAGCTAGCTAGCGTTTTAA
AAGGTGGTGTTGGCTCCCGTAT
E-11F
ATCCGTCACACCTGCTCTGTAAGGGGGGGGAATCGCTTTCGTCTTAAGAT
GACATGGTGTTGGCTCCCGTAT
E-12F
ATCCGTCACACCTGCTCTGCCGGACCATCCAATATCAGCTGTGGTGTTGG
CTCCCGTAT(59)
E-16F
ATCCGTCACACCTGCTCTATCCGTCACGCCTGCTCTATCCGTCACACCTG
CTCTGGTGTTGGCTCCCGTAT
E-17F
ATCCGTCACACCTGCTCTATCAAATGTGCAGATATCAAGACGATTTGTAC
AAGATGGTGTTGGCTCCCGTAT
E-18F
ATCCGTCACACCTGCTCTGTAGATGGCAAGGCATAAGCGTCCGGAACGAT
AGAATGGTGTTGGCTCCCGTAT
E-19F
ATCCGTCACACCTGCTCTGTAGATGGCAAGGCATAAGCGTCCGGAACGAT
AGAATGGTGTTGGCTCCCGTAT
E-5R
ATACGGGAGCCAACACCACCTTTTAAAACGCTAGCTAGCTTAGTCCATTC
CACCAGAGCAGGTGTGACGGAT
E-11R
ATACGGGAGCCAACACCATGTCATCTTAAGACGAAAGCGATTCCCCCCCC
TTACAGAGCAGGTGTGACGGAT
E-12R
ATACGGGAGCCAACACCACAGCTGATATTGGATGGTCCGGCAGAGCAGGT
GTGACGGAT
E-16R
ATACGGGAGCCAACACCAGAGCAGGTGTGACGGATAGAGCAGGCGTGACG
GATAGAGCAGGTGTGACGGAT
E-17R
ATACGGGAGCCAACACCATCTTGTACAAATCGTCTTGATATCTGCACATT
TGATAGAGCAGGTGTGACGGAT
E-18R
ATACGGGAGCCAACACCATTCTATCGTTCCGGACGCTTATGCCTTGCCAT
CTACAGAGCAGGTGTGACGGAT
E-19R
ATACGGGAGCCAACACCATTCTATCGTTCCGGACGCTTATGCCTTGCCAT
CTACAGAGCAGGTGTGACGGAT

Listeriolysin (a Surface Protein on Listeria monocytogenes)

LO-10F
ATCCGTCACACCTGCTCTGCCGGACCATCCAATATCAGCTGTGGTGTTGG
CTCCCGTAT
LO-11F
ATCCGTCACACCTGCTCTGGTGGAATGGACTAAGCTAGCTAGCGTTTTAA
AAGGTGGTGTTGGCTCCCGTAT
LO-13F
ATCCGTCACACCTGCTCTTAAAGTAGAGGCTGTTCTCCAGACGTCGCAGG
AGGATGGTGTTGGCTCCCGTAT
LO-15F
ATCCGTCACACCTGCTCTGTAGATGGCAAGGCATAAGCGTCCGGAACGAT
AGAATGGTGTTGGCTCCCGTAT
LO-16F
ATCCGTCACACCTGCTCTGTAGATGGCAAGGCATAAGCGTCCGGAACGAT
AGAATGGTGTTGGCTCCCGTAT
LO-17F
ATACGGGAGCCAACACCA
CAGCTGATATTGGATGGTCCGGCAGAGCAGGTGTGACGGAT
LO-19F
ATCCGTCACACCTGCTCTTGGGCAGGAGCGAGAGACTCTAATGGTAAGCA
AGAATGGTGTTGGCTCCCGTAT
LO-20F
ATCCGTCACACCTGCTCTCCAACAAGGCGACCGACCGCATGCAGATAGCC
AGGTTGGTGTTGGCTCCCGTAT
LO-10R
ATACGGGAGCCAACACCACAGCTGATATTGGATGGTCCGGCAGAGCAGGT
GTGACGGAT
LO-11R
ATACGGGAGCCAACACCACCTTTTAAAACGCTAGCTAGCTTAGTCCATTC
CACCAGAGCAGGTGTGACGGAT
LO-13R
ATACGGGAGCCAACACCATCCTCCTGCGACGTCTGGAGAACAGCCTCTAC
TTTAAGAGCAGGTGTGACGGAT
LO-15R
ATACGGGAGCCAACACCATTCTATCGTTCCGGACGCTTATGCCTTGCCAT
CTACAGAGCAGGTGTGACGGAT
LO-16R
ATACGGGAGCCAACACCATTCTATCGTTCCGGACGCTTATGCCTTGCCAT
CTACAGAGCAGGTGTGACGGAT
LO-17R
ATCCGTCACACCTGCTCTGCCGGACCATCCAATATCAGCTGTGGTGTTGG
CTCCCGTAT
LO-19R
ATACGGGAGCCAACACCATTCTTGCTTACCATTAGAGTCTCTCGCTCCTG
CCCAAGAGCAGGTGTGACGGAT
LO-20R
ATACGGGAGCCAACACCAACCTGGCTATCTGCATGCGGTCGGTCGCCTTG
TTGGAGAGCAGGTGTGACGGAT

### Listeriolysin (Alternate Form of Listeria Surface Protein Designated “Pest-Free”)

LP-3F
ATCCGTCACACCTGCTCTGTAGATGGCAAGGCATAAGCGTCCGGAACGAT
AGAATGGTGTTGGCTCCCGTAT
LP-11F
ATCCGTCACACCTGCTCTAACCAAAAGGGTAGGAGACCAAGCTAGCGATT
TGGATGGTGTTGGCTCCCGTAT
LP-13F
ATCCGTCACACCTGCTCTGCCGGACCATCCAATATCAGCT
GTGGTGTTGGCTCCCGTAT
LP-14F
ATCCGTCACACCTGCTCTGAAGCCTAACGGAGAAGATGGCCCTACTGCCG
TAGGTGGTGTTGGCTCCCGTAT
LP-15F
ATCCGTCACACCTGCTCTACTAAACAAGGGCAAACTGTAAACACAGTAGG
GGCGTGGTGTTGG
CTCCCGTAT
LP-17F
ATCCGTCACACCTGCTCTGGTGTTGGCTCCCGTATAGCTTGGCTCCCGTA
TGGTGTTGGCTCCCGTAT
LP-18F
ATCCGTCACACCTGCTCTGTCGCGATGATGAGCAGCAGCGCAGGAGGGAG
GGGGTGGTGTTGGCTCCCGTAT
LP-20F
ATCCGTCACACCTGCTCTGATCAGGGAAGACGCCAACACTGGTGTTGGCT
CCCGTAT
LP-3R
ATACGGGAGCCAACACCATTCTATCGTTCCGGACGCTTATGCCTTGCCAT
CTACAGAGCAGGTGTGACGGAT
LP-11R
ATACGGGAGCCAACACCATCCAAATCGCTAGCTTGGTCTCCTACCCTTTT
GGTTAGAGCAGGTGTGACGGAT
LP-13R
ATACGGGAGCCAACACCACAGCTGATATTGGATGGTCCGGCAGAGCAGGT
GTGACGGAT
LP-14R
ATACGGGAGCCAACACCACCTACGGCAGTAGGGCCATCTTCTCCGTTAGG
CTTCAGAGCAGGTGTGACGGAT
LP-15R
ATACGGGAGCCAACACCACGCCCCTACTGTGTTTACAGTTTGCCCTTGTT
TAGTAGAGCAGGTGTGACGGAT
LP-17R
ATACGGGAGCCAACACCATACGGGAGCCAAGCTATACGGGAGCCAACACC
AGAGCAGGTGTGACGGAT
LP-18R
ATACGGGAGCCAACACCACCCCCTCCCTCCTGCGCTGCTGCTCATCATCG
CGACAGAGCAGGTGTGACGGAT
LP-20R
ATACGGGAGCCAACACCAGTGTTGGCGTCTTCCCTGATCAGAGCAGGTGT
GACGGAT

Salmonella typhimurium Lipopolysaccharide (LPS)

St-7F
ATCCGTCACACCTGCTCTGTCCAAAGGCTACGCGTTAACGTGGTGTTGGC
TCCCGTAT
St-10F
ATCCGTCACACCTGCTCTGGAGCAATATGGTGGAGAAACGTGGTGTTGGC
TCCCGTAT
St-11F
ATCCGTCACACCTGCTCTGCCGGACCATCCAATATCAGCTGTGGTGTTGG
CTCCCGTAT
St-15F
ATCCGTCACACCTGCTCTGAACAGGATAGGGATTAGCGAGTCAACTAAGC
AGCATGGTGTTGGCTCCCGTAT
St-16F
ATCCGTCACACCTGCTCTGGCGGACAGGAAATAAGAATGAACGCAAAATT
TATCTGGTGTTGGCTCCCGTAT
St-18F
ATCCGTCACACCTGCTCTACGCAACGCGACAGGAACATTCATTATAGAAT
GTGTTGGTGTTGGCTCCCGTAT
St-19F
ATCCGTCACACCTGCTCTCGGCTGCAATGCGGGAGAGTAGGGGGGAACCA
AACCTGGTGTTGGCTCCCGTAT
St-20F
ATCCGTCACACCTGCTCTATGACTGGAACACGGGTATCGATGATTAGATG
TCCTTGGTGTTGGCTCCCGTAT
St-7R
ATACGGGAGCCAACACCACGTTAACGCGTAGCCTTTGGACAGAGCAGGTG
TGACGGAT
St-10R
ATACGGGAGCCAACACCACGTTTCTCCACCATATTGCTCCAGAGCAGGTG
TGACGGAT
St-11R
ATACGGGAGCCAACACCACAGCTGATATTGGATGGTCCGGCAGAGCAGGT
GTGACGGAT
St-15R
ATACGGGAGCCAACACCATGCTGCTTAGTTGACTCGCTAATCCCTATCCT
GTTCAGAGCAGGTGTGACGGAT
St-16R
ATACGGGAGCCAACACCAGATAAATTTTGCGTTCATTCTTATTTCCTGTC
CGCCAGAGCAGGTGTGACGGAT
St-18R
ATACGGGAGCCAACACCAACACATTCTATAATGAATGTTCCTGTCGCGTT
GCGTAGAGCAGGTGTGACGGAT
St-19R
ATACGGGAGCCAACACCAGGTTTGGTTCCCCCCTACTCTCCCGCATTGCA
GCCGAGAGCAGGTGTGACGGAT
St-20R
ATACGGGAGCCAACACCAAGGACATCTAATCATCGATACCCGTGTTCCAG
TCATAGAGCAGGTGTGACGGAT

### BRIEF DESCRIPTION OF THE DRAWINGS

FIG. 1. is a schematic illustration that illustrates a comparison of possible nucleic acid FRET assay formats.

FIGS. 2A. and 2B. are line graphs mapping relative fluorescence intensity against the concentration of surface protein from L. donovani from various freeze-dried and reconstituted competitive FRET-aptamer assays.

FIGS. 3A. and 3B. are “lights on” competitive FRET-aptamer spectra and a line graph for E. coli bacteria using aptamers generated against various components of lipopolysaccharide (LPS) such as the rough core (Ra) antigen and the 2-keto-3-deoxyoctanate (KDO) antigen.

FIGS. 4A. and 4B. are “lights on” competitive FRET-aptamer spectra and a bar graph for Enterococcus faecalis bacteria using aptamers generated against lipoteichoic acid.

FIGS. 5A. and 5B. are “lights off” competitive FRET-aptamer spectra and line graphs in response to increasing amounts of a foot-and-mouth disease (FMD) aphthovirus surface peptide.

FIGS. 6A. and 6B. are “lights on” competitive FRET-aptamer spectra and FIG. 6C. is a line graph in response to increasing amounts of methylphosphonic acid (MPA; an organophosphorus (OP) nerve agent breakdown product).

FIGS. 7A and 7B. are Sephadex G25 size-exclusion column profiles of complexes of Alexa Fluor (AF) 546-dUTP-labeled competitive FRET-aptamers bound to BHQ-2-amino-MPA (quencher-labeled target). The fractions with the highest absorbance at 260 nm (DNA aptamer), 555 nm (AF 546), and 579 nm (BHQ-2) were pooled and used in the competitive assay for unlabeled MPA, because these fractions contain the FRET-aptamer-quencher-labeled target complexes.

### DETAILED DESCRIPTION OF THE PREFERRED EMBODIMENT

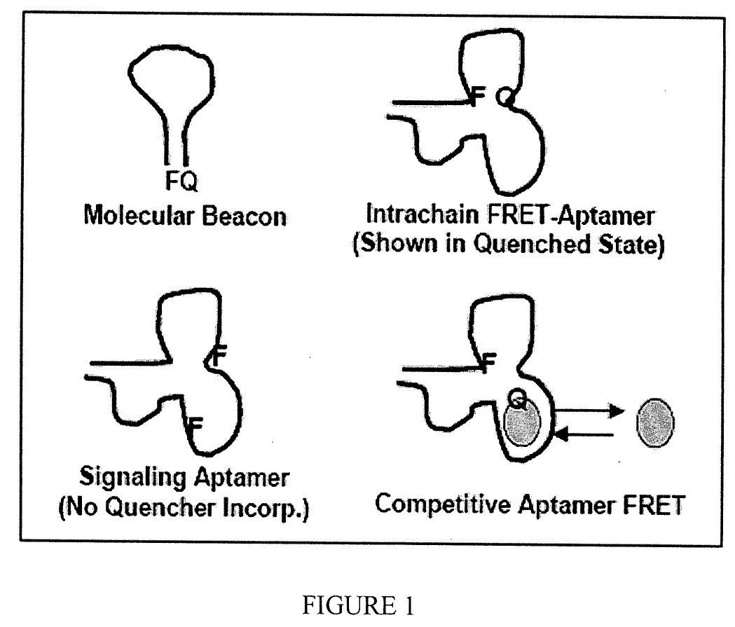

Referring to the figures, FIG. 1. provides a comparison of possible nucleic acid FRET assay formats. It illustrates how the competitive aptamer FRET scheme differs from other oligonucleotide-based FRET assay formats. Upper left is a molecular beacon (10) which may or may not be an aptamer, but is typically a short oligonucleotide used to hybridize to other DNA or RNA molecules and exhibit FRET upon hybridizing. Molecular beacons are only labeled with F and Q at the ends of the DNA molecule. Lower left is a signaling aptamer (12), which does not contain a quencher molecule, but relies upon fluorophore self-quenching or weak intrinsic quenching of the DNA or RNA to achieve limited FRET. Upper right is an intrachain FRET-aptamer (14) containing F and Q molecules built into the interior structure of the aptamer. Intrachain FRET-aptamers are naturally selected and characterized by the processes described herein. Lower right shows a competitive aptamer FRET (16) motif in which the aptamer container either F or Q and the target molecule (18) is labeled with the complementary F or Q. Introduction of unlabeled target molecules (20) then shifts the equilibrium so that some labeled target molecules are liberated from the labeled aptamer and modulate the fluorescence level of the solution up or down thereby achieving FRET. A target analyte (20) is either unlabeled or labeled with a quencher (Q). F and Q can be switched from placement in the aptamer to placement in the target analyte and vice versa.

F-labeled or Q-labeled aptamers (labeled by the polymerase chain reaction (PCR), asymmetric PCR (to produce a predominately single-stranded amplicon) using Taq, Deep Vent Exo⁻ or other heat-resistant DNA polymerases, or other enzymatic incorporation of F-NTPs or Q-NTPs) may be used in competitive or displacement type assays in which the fluorescence light levels change proportionately in response to the addition of various levels of unlabeled analyte which compete to bind with the F-labeled or Q-labeled analytes.

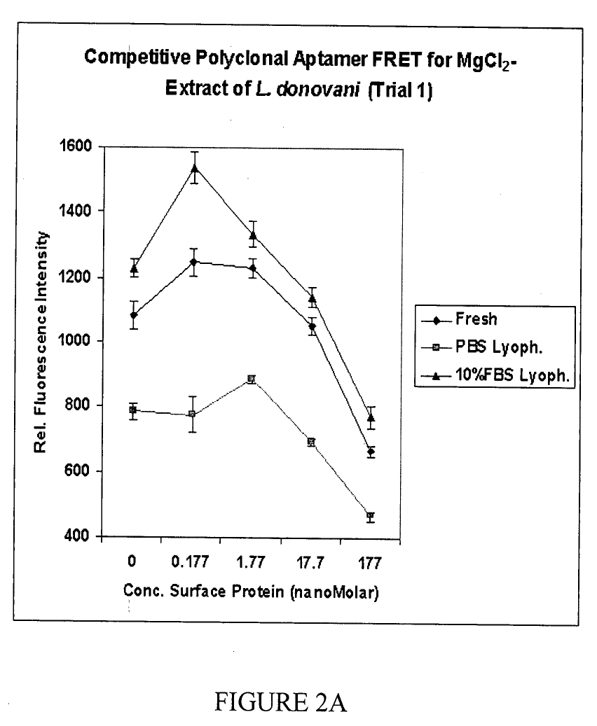

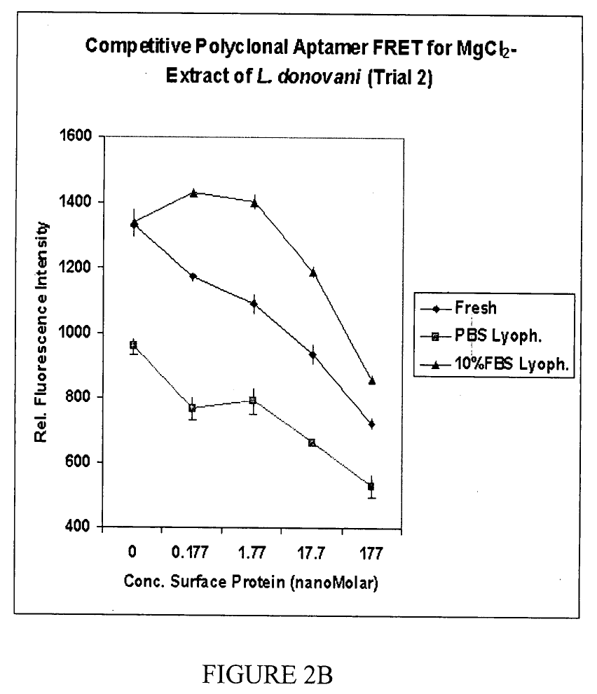

Competitive aptamer-FRET assays may be used for the detection and quantitation of small molecules (<1,000 daltons) including pesticides, acetylcholine (ACh), organophosphate (“OP”) nerve agents such as sarin, soman, and VX, OP nerve agent breakdown products such as MPA, isopropyl-MPA, ethylmethyl-MPA, pinacolyl-MPA, etc., acetylcholine (ACh), acyl homoserine lactone (AHL) and other quorum sensing (QS) molecules natural and synthetic amino acids and their derivatives (e.g., histidine, histamine, homocysteine, DOPA, melatonin, nitrotyrosine, etc.), short chain proteolysis products such as cadaverine, putrescine, the polyamines spermine and spermidine, nitrogen bases of DNA or RNA, nucleosides, nucleotides, and their cyclical isoforms (e.g., cAMP and cGMP), cellular metabolites (e.g., urea, uric acid), pharmaceuticals (therapeutic drugs), drugs of abuse (e.g., narcotics, hallucinogens, gamma-hydroxybutyrate, etc.), cellular mediators (e.g., cytokines, chemokines, immune modulators, neural modulators, inflammatory modulators such as prostaglandins, etc.), or their metabolites, explosives (e.g., trinitrotoluene) and their breakdown products or byproducts, peptides and their derivatives, macromolecules including proteins (such as bacterial surface proteins from Leishmania donovani, See FIGS. 2A and 2B), glycoproteins, lipids, glycolipids, nucleic acids, polysaccharides, lipopolysaccharides (LPS), and LPS components (e.g., ethanolamine, glucosamine, LPS-specific sugars, KDO, rough core antigens, etc.), viruses, whole cells (bacteria and eukaryotic cells, cancer cells, etc.), and subcellular organelles or cellular fractions.

If the target molecule is a larger water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTP's that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule, then exposure of the nascent F-labeled and Q-labeled DNA or RNA random library to the target may be done by immobilizing the target. The small molecule can be immobilized on a column, membrane, plastic or glass bead, magnetic bead, quantum dot, or other matrix. If no functional group is available on the small molecule for immobilization, the target can be immobilized by the Mannich reaction (formaldehyde-based condensation reaction) on a PharmaLink™ column. Elution of bound DNA from the small molecule affinity column, membrane, beads or other matrix by use of 0.2-3.0M sodium acetate at a pH of between 3 and 7.

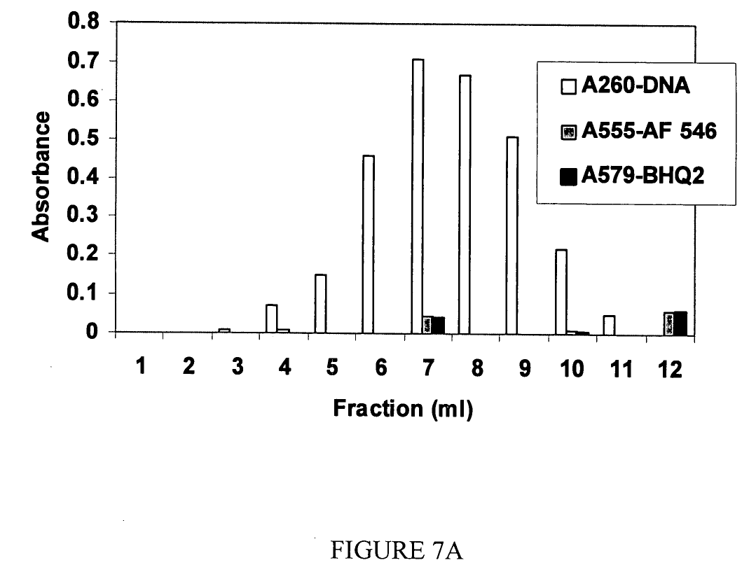

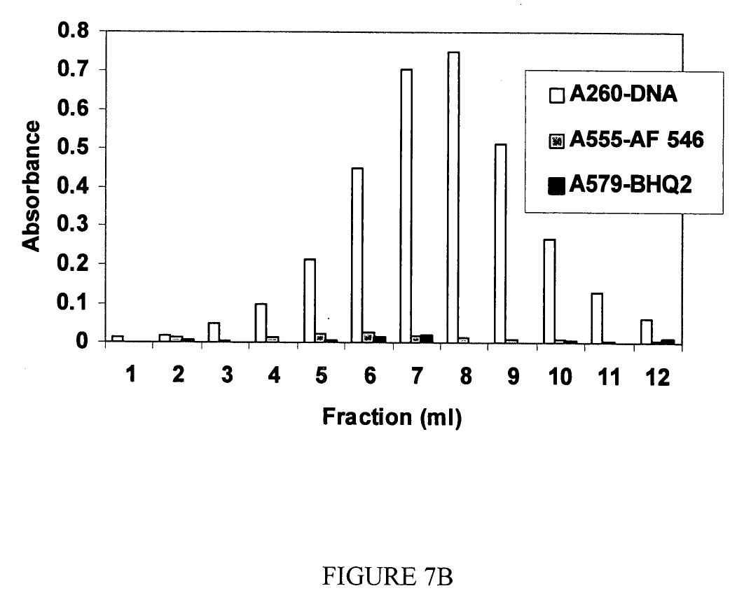

These can be separated from the non-binding doped DNA molecules by running the aptamer-protein aggregates (or selected aptamers-protein aggregates) through a size exclusion column, by means of size-exclusion chromatography using Sephadex™ or other gel materials in the column. Since they vary in weight due to variations in aptamers sequences and degree of labeling, they can be separated into fractions with different fluorescence intensities. Purification methods such as preparative gel electrophoresis are possible as well. Small volume fractions (<1 mL) can be collected from the column and analyzed for absorbance at 260 nm and 280 nm which are characteristic wavelengths for DNA and proteins. In addition, the characteristic absorbance wavelengths for the fluorophore and quencher (FIGS. 7A and 7B) should be monitored. The heaviest materials come through a size-exclusion column first. Therefore, the DNA-protein complexes will come out of the column before either the DNA or protein alone.

Means of separating FRET-aptamer-target complexes from solution by alternate techniques (other than size-exclusion chromatography) include, without limitation, molecular weight cut off spin columns, dialysis, analytical and preparative gel electrophoresis, various types of high performance liquid chromatography (HPLC), thin layer chromatography (TLC), and differential centrifugation using density gradient materials.

The optimal (most sensitive or highest signal to noise ratio) FRET-aptamers among the bound class of FRET-aptamer-target complexes are identified by assessment of fluorescence intensity for various fractions of the FRET-aptamer-target class. The separated DNA-protein complexes will exhibit the highest absorbance at established wavelengths, such as 260 nm and 280 nm. The fractions showing the highest absorbance at the given wavelengths, such as 260 nm and 280 nm, are then further analyzed for fluorescence and those fractions exhibiting the greatest fluorescence are selected for separation and sequencing.

These similar FRET-aptamers may be further separated using techniques such as ion pair reverse-phase high performance liquid chromatography, ion-exchange chromatography (IEC, either low pressure or HPLC versions of IEC), thin layer chromatography (TLC), capillary electrophoresis, or similar techniques.

The final FRET aptamers are able to act as one-step “lights on” or “lights off” binding and detection components in assays.

Intrachain FRET-aptamers that are to be used in assays with long shelf-lives may be lyophilized (freeze-dried) and reconstituted.

FIGS. 2A. and 2B. are line graphs mapping the fluorescence intensity of the DNA aptamers against the concentration of the surface protein. The figures present results from two independent trials of a competitive aptamer-FRET assay involving fluorophore-labeled DNA aptamers and surface extracted proteins from Leishmania donovani bacteria. In this type of assay, the fluorescence intensity decreases as a function of increasing analyte concentration, and is thus referred to as a “lights off” assay. If the fluorescence intensity increases as a function of increasing analyte concentration, then it is referred to as a “lights on” assay. Also shown are translations of the assay curve up or down due to lyophilization (freeze-drying) in the absence or presence of 10% fetal bovine serum (FBS). Error bars represent the standard deviations of the mean for three measurements.

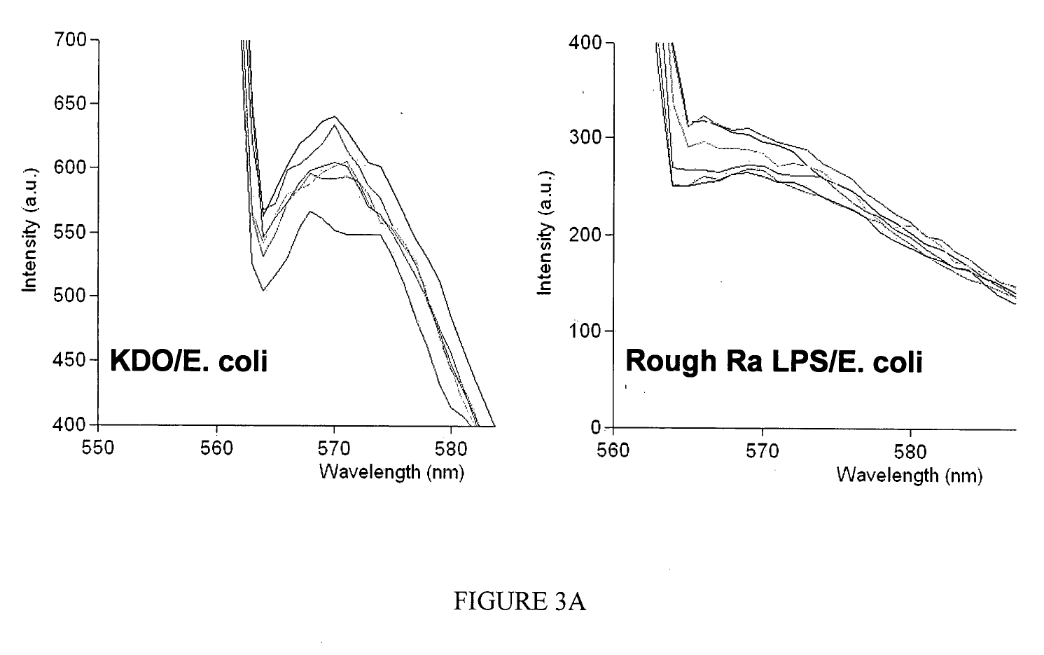

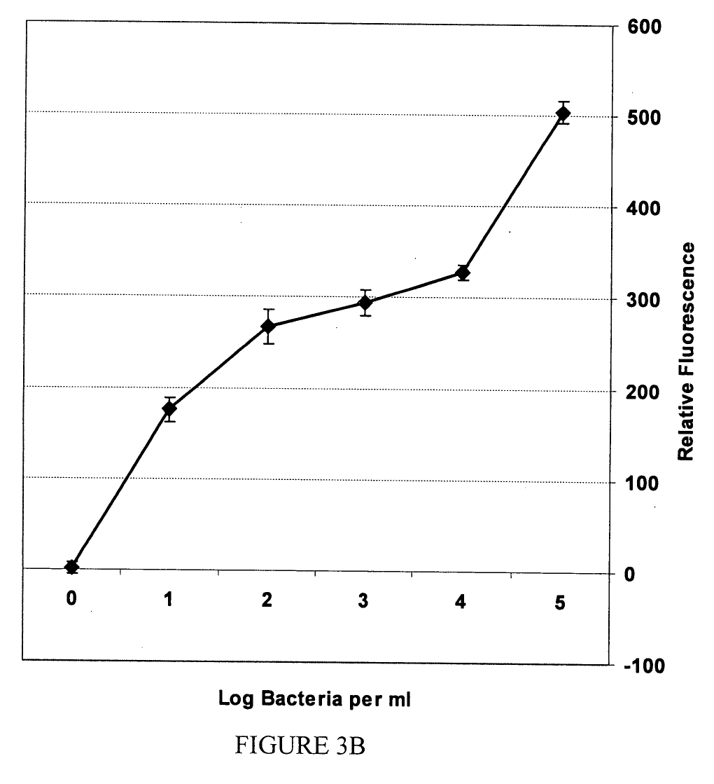

FIGS. 3A. and 3B. are FRET fluorescence spectra and line graphs generated as a function of live E. coli (Crooks strain, ATCC No. 8739) concentration using LPS component competitive FRET-aptamers. Error bars represent the standard deviations of the mean for four measurements.

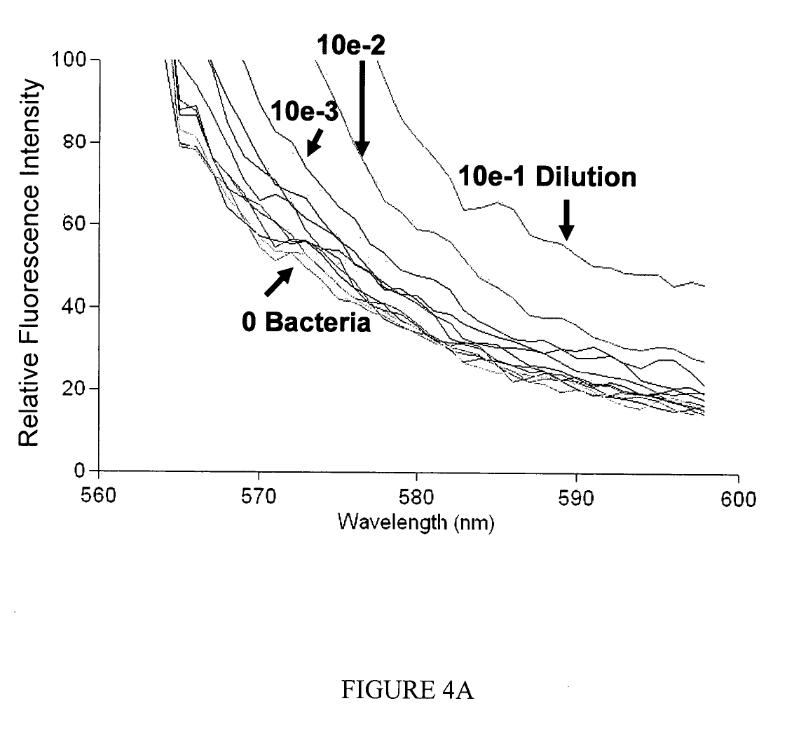

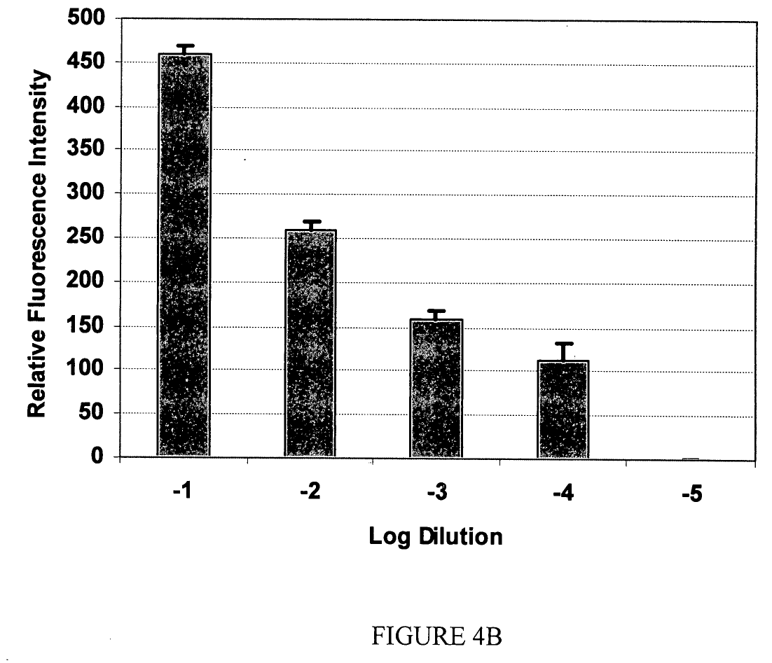

FIGS. 4A. and 4B. are FRET fluorescence spectra and line graphs generated as a function of live Enterococcus faecalis concentration using lipoteichoic acid (TA) competitive FRET-aptamers. Error bars represent the standard deviations of the mean for four measurements.

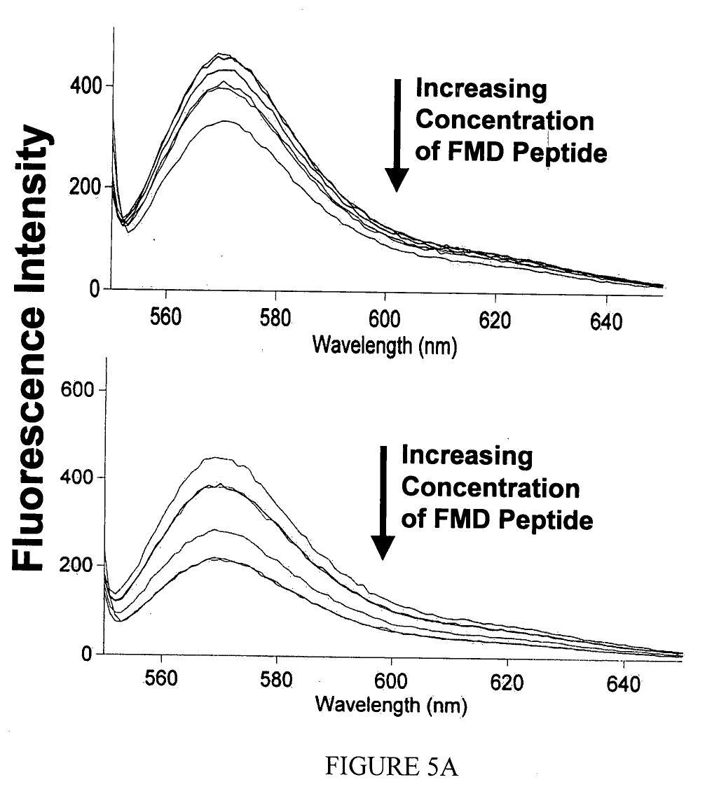

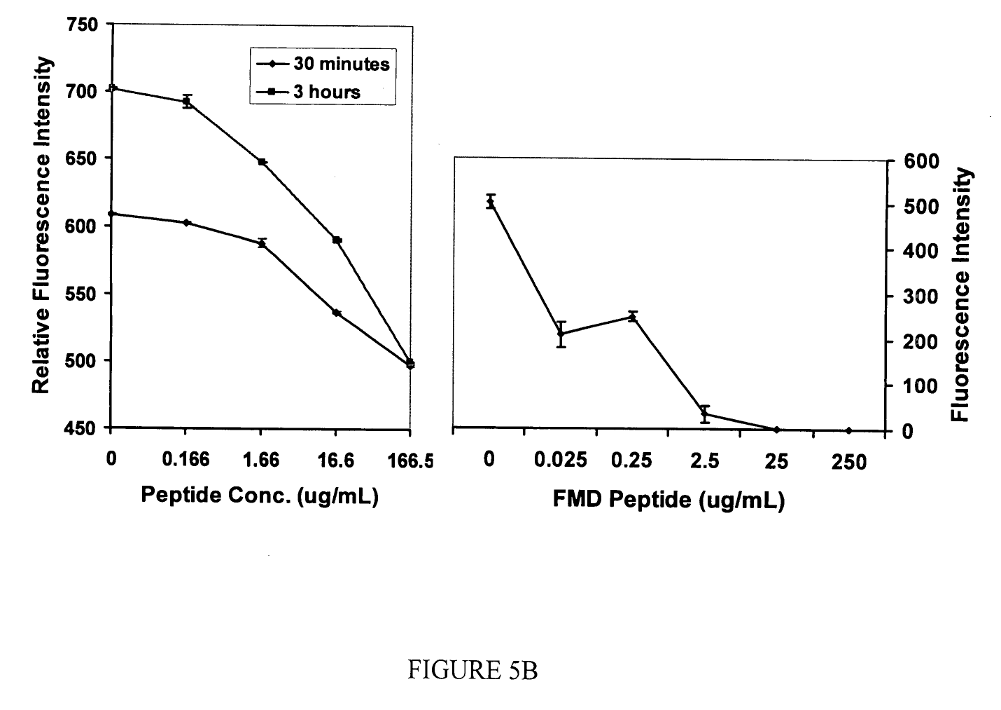

FIGS. 5A. and 5B. are FRET fluorescence spectra and line graphs generated as a function of Foot-and-Mouth Disease (FMD) peptide concentration using FMD peptide competitive FRET-aptamers. Error bars represent the standard deviations of the mean for four measurements.

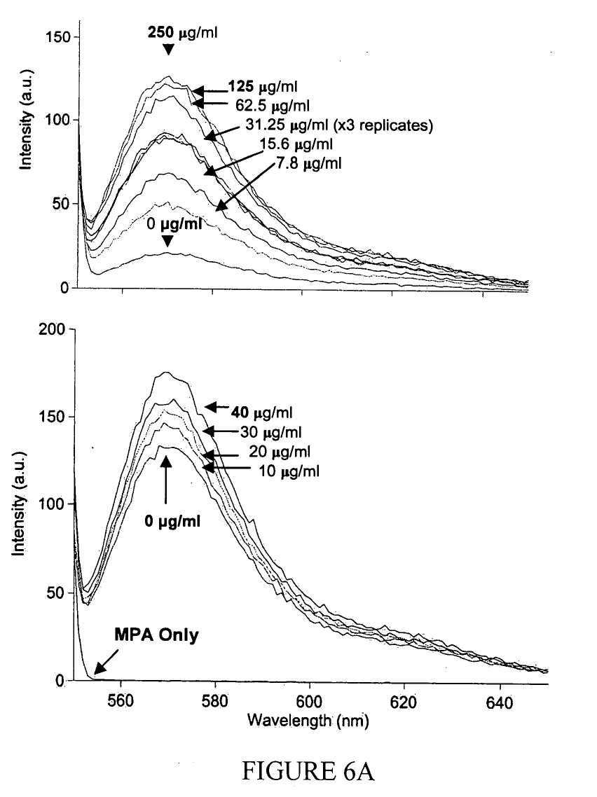

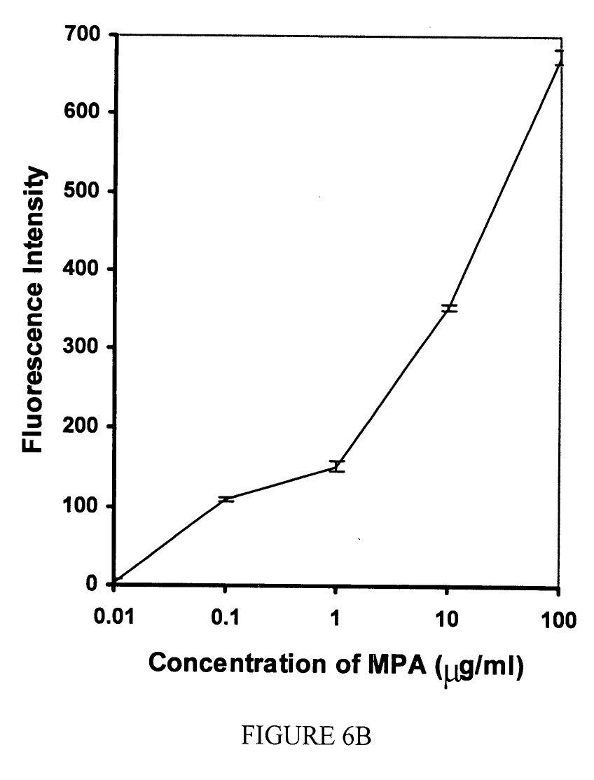

FIGS. 6A. and 6B. are FRET fluorescence spectra, and FIG. 6C. is a line graph, all generated as a function of methylphosphonic acid (MPA; OP nerve agent degradation product) concentration using MPA competitive FRET-aptamers to represent possible FRET-aptamer assays for MPA and OP nerve agents such as pesticides, sarin, soman, VX, etc. Error bars represent the standard deviations of the mean for four measurements.

FIGS. 7A. and 7B. are two independent Sephadex™ G25 elution profiles for BHQ-2-amino-MPA-AF 546-MPA aptamer complex based on absorbance peaks characteristic of the aptamer (260 nm), fluorophore (555 nm), and quencher (579 nm) to assess the optimal fraction for competitive FRET-aptamer assay of MPA shown in FIG. 6. Similar elution profiles can be expected for all such soluble targets when the target is quencher-labeled and complexed to a fluorophore-labeled aptamer.

### Example 1

### Competitive Aptamer-FRET Assay for Surface Proteins Extracted from Bacteria (L. donovani)

In this example, surface proteins from heat-killed Leishmania donovani were extracted with 3 M MgCl₂ overnight at 4° C. These proteins were then linked to tosyl-magnetic microbeads and used in a standard SELEX aptamer generation protocol. After 5 rounds of SELEX, the aptamer population was “doped” during the standard PCR reaction with 3 uM fluorescein-dUTP and purified on 10 kD molecular weight cut off spin columns. Some of the L. donovani surface proteins were then labeled with dabcyl-NHS ester and purified on a PD-10 (Sephadex G25) column. The dabcyl-labeled surface proteins were combined with the fluorescein-labeled aptamer population so as to produce a 1:1 fluorescein-aptamer:dabcyl-protein ratio. Thereafter, unlabeled L. donovani surface proteins were introduced into the assay system to compete with the labeled proteins for binding to the aptamers, thereby producing the “lights off” FRET assay results depicted in FIGS. 2A and 2B (fresh assay results, solid line). The assays were also examined following lyophilization (freeze drying) and reconstitution (rehydration) in the presence or absence of 10% fetal bovine serum (FBS) as a possible preservative with the results shown in FIGS. 2A and 2B. The DNA sequences of several of these candidate Leishmania aptamers are given in SEQ IDs XX-XX.

### Example 2

### Competitive Fret-Aptamer Assay for E. Coli in Environmental Water Samples or Foods Using LPS Component Aptamers

E. coli, especially the enterohemorrhagic strains such as O157:H7 which produce Verotoxin or Shiga toxins, are of concern in environmental water samples and foods. Their rapid detection (within minutes) with ultrasensitivity is important in protecting swimmers as well as those consuming water and foods. In this example, aptamers were generated against whole LPS from E. coli O111:B4 and its components such as glucosamine, KDO, and the rough mutant core antigen (Ra; lacking the outer oligosaccharide chains). In the case of glucosamine, the free primary amine in its structure enabled conjugation to tosyl-magnetic beads. KDO antigen was immobilized onto amine-conjugated magnetic beads via its carboxyl group and the bifunctional linker EDC. The rough Ra core antigen and whole LPS were linked to amine-magnetic beads via reductive amination using sodium periodate to oxidize the saccharides to aldehydes followed by the use of sodium cyanoborohydride for reductive amination as will be clear to anyone skilled in the art. Once immobilized the target-magnetic beads were used for aptamer affinity selection from a random library of 72 base aptamers (randomized 36mer flanked by known 18mer primer regions). After 5 rounds of aptamer selection and amplification, the various LPS component aptamer populations were subjected to 10 rounds of PCR in the presence of Alexa Fluor (AF) 546-14-dUTP (Invitrogen), then heated to 95° C. for 5 minutes and added to heat-killed E. coli O157:H7 (Kirkegaard Perry Laboraties, Inc., Gaithersburg, Md.) and used in competitive FRET-aptamer assays with various concentrations of unlabeled live E. coli (Crooks strain, ATCC No. 8739) resulting in the FRET spectra and line graphs shown in FIGS. 3A and 3B. Candidate DNA aptamer sequences for detection of LPS 0111 and LPS components or associated E. coli and other Gram negative bacteria are given in SEQ ID Nos. XX-XX.

### Example 3

### Competitive FRET-Aptamer Assay for Enterococci in Environmental Water Samples

Gram positive enterococci, such as Enterococcus faecalis, are also indicators of fecal contamination of environmental water, recreational waters, or treated wastewater (effluent from sewage treatment plants). Water testers desire to detect the presence of these bacteria rapidly (within minutes) and with great sensitivity. In this example, aptamers were generated against whole lipoteichoic acid (TA; teichoic acid). TA from E. faecalis was immobilized on magnetic beads by reductive amination using sodium periodate to first oxidize saccharides into aldehydes followed by reductive amination using amine-magnetic beads and sodium cyanoborohydride as will be known to anyone skilled in the art. Once immobilized the target-magnetic beads were used for aptamer affinity selection from a random library of 72 base aptamers (randomized 36mer flanked by known 18mer primer regions). After 5 rounds of aptamer selection and amplification, the TA aptamer population was subjected to 10 rounds of PCR in the presence of AF 546-14-dUTP (Invitrogen), then heated to 95° C. for 5 minutes and added to live E. faecalis. The complexes were purified by centrifugation and washing and used in competitive FRET-aptamer assays with various concentrations of unlabeled live E. faecalis resulting in the FRET spectra and bar graphs shown in FIGS. 4A. and 4B. Candidate DNA aptamer sequences for detection of lipoteichoic acid (TA) and associated enterococi or other Gram positive bacteria are given in SEQ ID Nos. XX-XX.

### Example 4

### Detection of Foot-And-Mouth (FMD) Disease or Other Highly Communicable Viruses Among Animal or Human Populations

FMD has not existed in the United States for decades, but if it were reintroduced via agricultural bioterrorism or accidental means, it could cripple the multi-billion dollar livestock industry. Hence, rapid detection of FMD in the field (on farms) is of great value in quarantining infected animals or farms and limiting the spread of FMD. Likewise, epidemiologists have many uses for rapid field detection and identification of viruses and other microbes such as influenzas, potential small pox outbreaks, etc. which FRET-aptamer assays could satisfy. A highly conserved peptide from the VP1 structural protein of O-type FMD, which is widely distributed throughout the world, was chosen as the aptamer development target. The peptide had the following primary amino acid sequence: RHKQKIVAPVKQLL. This sequence corresponds to amino acids 200 through 213 of 16 different O-type FMD viruses and represents a neutralizable antigenic region wherein antibodies are known to bind. The FMD peptide was immobilized on tosyl-magnetic beads via the three lysine residues in its structure. Once immobilized the target-magnetic beads were used for aptamer affinity selection from a random library of 72 base aptamers (randomized 36mer flanked by known 18mer primer regions). After 5 rounds of aptamer selection and amplification, the FMD (peptide) aptamer populations were subjected to 10 rounds of PCR in the presence of Alexa Fluor (AF) 546-14-dUTP (Invitrogen), then heated to 95° C. for 5 minutes and added to their BHQ-2-labeled-peptide target. The complexes were purified by size-exclusion chromatography over Sephadex G25 and used in competitive FRET-aptamer assays with various concentrations of unlabeled FMD peptide resulting in the FRET spectra and line graphs shown in FIGS. 5A and 5B. Candidate DNA aptamer sequences for detection of the FMD peptide and associated strains of FMD virus are given in SEQ ID Nos. XX-XX.

### Example 5

### Detection of Organophosphorus (OP) Nerve Agent, Pesticides, and Acetylcholine (ACh)

The use of OP nerve agents on Iraqi Kurds in the late 1980's followed by the 1995 use of sarin in a Japanese subway underscore the need for rapid and sensitive detection of OP nerve agents such as FRET-aptamer assays might provide. In addition, there is a desire in the agricultural industry to detect pesticides (also OP nerve agents) on the surfaces of fruits and vegetables in the field or in grocery stores. Finally, aptamers that bind and detect acetylcholine (ACh) may be of value in determining the impact of OP nerve agents on acetylcholinesterase (AChE) activity. Candidate aptamer sequences for the nerve agent soman, methylphosphonic acid (MPA, a common nerve agent hydrolysis product), the pesticides diazinon and malathion, and ACh are given in SEQ ID Nos. XX-XX. Amino-MPA and para-aminophenyl-soman were immobilized on tosyl-magnetic beads and used for aptamer selection. ACh and the pesticides were immobilized onto PharmaLink™ (Pierce Chemical Co.) affinity columns by the Mannich formaldehyde condensation reaction and used for aptamer selection. The polyclonal or monoclonal candidate MPA aptamers were labeled with AF 546-14-dUTP by 10 rounds of conventional PCR or 20 rounds of asymmetric as appropriate with Deep Vent Exo⁻ polymerase and then complexed to BHQ-2-amino-MPA. The complexes were purified by size-exclusion chromatography over Sephadex G-15 and used to generate FRET spectra and line graphs as a function of unlabeled MPA as shown in FIGS. 6A., 6B., and 6C.

Other potential examples of uses for competitive FRET-aptamer assays include, but are not limited to:

1) Detection and quantitation of quorum sensing (QS) molecules such as acyl homoserine lactones (AHLs such as N-Decanoyl-DL-Homoserine Lactone; SEQ ID Nos. XX-XX), which communicate between many Gram negative bacteria such as Pseudomonads to signal proliferation and the induction of virulence factors, thereby leading to disease.
2) Detection and quantitation of botulinum toxins (BoNTs) for determination of the presence of biological warfare or bioterrorism agents (SEQ ID Nos. XX-XX) and Clostridium botulinum in vivo.
3) Detection and quantitation of Campylobacter jejuni and related Campylobacter species (SEQ ID Nos. XX-XX) in foods and water to prevent foodborne or waterborne illness outbreaks (add 2006 JCLA paper reference here).
4) Detection and quantitation of poly-D-glutamic acid (PDGA; SEQ ID Nos. XX-XX) from vegetative forms of pathogenic Bacillus anthracis or other similar encapsulated bacteria in vivo or in the environment to rapidly diagnose biological warfare or bioterrorist activity and provide intervention.
5) Detection and quantitation of Bacillus thuringiensis bacterial endospores in the environment to assist in biological warfare or bioterrorism detection field trials or forensic work.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

We claim:

1. A method of using a competitive type assay, comprising:
running an assay;
incorporating F-labeled or Q-labeled aptamers, wherein said aptamers are labeled with said F's and Q's located on the interior portion of said aptamer;
adding a volume of unlabeled analyte, wherein said analyte competes to bind with said F-labeled or Q-labeled analytes;
wherein fluorescence light levels change proportionately in response to the amount of said volume of unlabeled analyte; and
wherein said competitive type assay detects molecules selected from the group consisting of: pesticides, OP nerve agents, OP nerve agent breakdown products, acetylcholine (ACh), acyl homoserine lactone (AHL) and other quorum sensing (QS) molecules, natural and synthetic amino acids and their derivatives, histidine, histamine, homocysteine, DOPA, melatonin, nitrotyrosine, short chain proteolysis products, cadaverine, putrescine, polyamines, spermine, spermidine, nitrogen bases of DNA or RNA, nucleosides, nucleotides, nucleotide cyclical isoforms, cAMP, cGMP, cellular metabolites, urea, uric acid, pharmaceuticals, therapeutic drugs, illegal drugs, narcotics, hallucinogens, gamma-hydroxybutyrate (GHB), cellular mediators, cytokines, chemokines, immune modulators, neural modulators, inflammatory modulators, prostaglandins, prostaglandin metabolites, explosives, trinitrotoluene, explosive breakdown products or byproducts, peptides and their derivatives, such as poly-D-glutamic acid (PDGA) and similar bacterial capsule materials, macromolecules, proteins, bacterial surface proteins, glycoproteins, lipids, glycolipids, nucleic acids, polysaccharides, lipopolysaccharides or LPS components, lipoteichoc or teichoic acids, viruses, whole cells, spores or endospores, and subcellular organelles or cellular fractions.

2. The method of claim 1, further comprising:
immobilizing said small molecules on a column, membrane, plastic or glass bead, magnetic bead, quantum dot, or other matrix;
eluting immobilized aptamers from said column, membrane, plastic or glass bead, magnetic bead, or other matrix by use of 0.2-3.0 M sodium acetate at a pH of between 3 and 7.

3. The method of claim 1, wherein said detected molecules are quantified.

4. A method of using a competitive type assay, comprising:
running an assay; and
incorporating an aptamer, wherein said aptamer is selected from the SEQ Aptamers.

5. The method of claim 4, further comprising:
adding a volume of unlabeled analyte, wherein said analyte competes to bind with said F-labeled or Q-labeled analytes; and
wherein fluorescence light levels change proportionately in response to the amount of said volume of unlabeled analyte.

6. A method of using a competitive type assay, comprising:
running an assay;
incorporating an aptamer;
wherein said aptamer has a binding pocket; and
wherein said binding pocket is comprised of 3 to 6 nucleotides.

7. The method of claim 6 wherein said binding pocket is comprised of 3 or more nucleotides of a specific sequence or arrangement to confer the appropriate volume and conformation in 3-dimensional space to enable optimal binding to target molecules.

8. The method of claim 6 wherein said aptamer is selected from the SEQ Aptamers.

9. The method of claim 7 wherein said aptamer is selected from the SEQ Aptamers.
